(19) United States
(12) Patent Application Publication
(10) Pub. No.: US 2023/0201018 A1
(43) Pub. Date: Jun. 29, 2023

(54) ORTHOPEDIC BRACING SYSTEM AND METHOD OF USE

(71) Applicant: Step Ahead Medical LLC, Medford, NJ (US)

(72) Inventors: Rick Raimondo, Medford, NJ (US); John Dudley, West Chester, PA (US); Nadia Din, Barnegat, NJ (US); Charles Soltner, Mullica-Hill, NJ (US)

(21) Appl. No.: 18/177,389

(22) Filed: Mar. 2, 2023

Related U.S. Application Data
(63) Continuation of application No. PCT/US21/49211, filed on Sep. 7, 2021.
(60) Provisional application No. 63/108,314, filed on Oct. 31, 2020.
(60) Provisional application No. 63/074,514, filed on Sep. 4, 2020.

(51) Int. Cl. A61F 5/01; A61F 5/34

(52) U.S. Cl. CPC A61F 5/0111; A61F 5/0195; A61F 5/34

(57) ABSTRACT

A modular orthopedic bracing system is provided. The orthopedic bracing system includes a walking boot configured to cover at least the plantar aspect of the user's foot, a support detachably connectable to the walking boot and configured to suspend the weight of the user and an ankle-foot orthosis (“AFO”) detachably connectable to the walking boot. Also disclosed is a method for supporting an injured lower extremity, which includes the steps of attaching a first support member to the patient's leg, providing a second support member for an area at or beneath the foot and posterior portion of the lower leg, providing a first upward force on the patient's leg with the first support member, providing a second upward force on the foot with the second support member. By adjusting the amounts of the first upward force and the second upward force, the foot and lower leg, supported by the AFO, may be fully suspended, partially suspended or rest completely on the foot bed of the walking boot.

### RELATED APPLICATIONS

This application is a continuation of International Application No. PCT/US21/49211, filed Sep. 7, 2021, which claims priority to U.S. Provisional Patent Application No. 63/108,314, filed Oct. 31, 2020, and U.S. Provisional Patent Application No. 63/074,514, filed Sep. 4, 2020, the entire disclosures of which are hereby incorporated herein by reference.

### BACKGROUND

The teachings of this disclosure generally relate to an orthopedic bracing system and method for bracing a patient's leg throughout the period of recovery from an injury or surgery.

Certain types of injuries and surgeries require the patient's ankle, foot and lower leg to be immobilized at different stages and supported to facilitate recovery. It is also frequently necessary to reduce or eliminate the amount of weight being borne by the ankle or foot during the recovery period following an injury or surgery. Further, providing increased protection from the environment for the foot or ankle may also be required during the recovery period.

Currently available orthopedic devices generally only accomplish one of the three objectives. For example, an ankle-foot orthosis (“AFO”) will stabilize and support the foot and ankle in an optimal position, but will only offer limited protection of the foot and ankle from the environment and will not offload the weight of the user from the foot and ankle. For limb protection and off-loading, a combination of casts or walking boots with crutches, walkers, scooters and other devices must be used. Consequently, a patient may be required to purchase and use multiple devices over the course of his or her recovery.

Further, many orthopedic braces have to be custom fit to the user. This often requires a lengthy process that includes sending a patient to an outside lab for fabrication of the brace. The process is expensive, time consuming and frequently delays treatment for several weeks or more.

Another problem with existing bracing systems is compliance. Because the individual elements of a complete bracing system are not designed to work together in a way that is cost-effective and convenient for the patient and do not take into account the patient's needs at different stages of recovery, or even at different times of the day, many patients do not comply with the treatment instructions. Noncompliance or removal of the bracing system prematurely may result in harm to the foot, ankle and leg with, for instance, displacement of a fracture, delayed healing of a wound or a prolonged period of recovery and rehabilitation. For example, a walking boot is often used to protect an injured or post-operative foot and ankle without the expense of a custom-fit AFO. However, these boots often become dirty over the course of the day. They are large, cumbersome and frequently cause the lower limb to become hot and sweaty. For these reasons and others, patients often remove the boots during the day for relief and at night rather than bringing the unsanitary boot into bed. As a result, the foot and ankle are left unprotected and are not maintained at the proper angle intermittently during the day and during most of the night. This can lead to contractures or other injuries that may require more healing time, additional surgery and more extensive rehabilitation than if the ankle were braced in the correct position at all times. Patients sometimes also forget to put the walking boot back on before getting off the couch or out of bed, causing further harm. Noncompliance with the walking boot, removing it during the day and at night, is a prevalent problem.

Moreover, a patient's needs change over the course of recovery, but currently available devices are not designed to easily adapt. For example, in the beginning, a patient may not be able to bear any weight on an injured foot and crutches may be prescribed to offload the foot. As recovery progresses, the patient may only need to offload 50% of his or her weight, but it is difficult to know how to bear 50% of one's weight on crutches and so patients often just carry the crutches around with them, placing 100% of their weight plus the weight of the crutches on the injured foot.

What is needed is an orthopedic bracing system that can be adapted to fit a wide range of patients without the delay and expense of custom fitting and that can meet most or all of the patient's needs throughout the stages of recovery and throughout the patient's normal daily routine. Also needed is a device that can offload and protect a limb during the day and night even if the outer bulky shell, or walking boot, is removed.

### SUMMARY

The present disclosure provides a modular orthopedic bracing system, which includes an ankle-foot orthosis (“AFO”), a walking boot and a patellar tendon bearing brace (“PTB”). In certain embodiments, the PTB is replaced with a support that attaches to the patient's thigh and upper calf area.

The AFO can be configured to maintain a patient's foot, ankle and lower leg at the prescribed angle to allow healing and avoid, for example, contracture. The exterior shell of the AFO is formed from a rigid or semi-rigid material, e.g., plastic. The AFO has a base that attaches to and supports the patient's foot and an upright portion, which forms an angle with the base. The angle between the base and the upright portion may be generally perpendicular. The upright portion attaches to the patient's leg with, e.g., straps. The AFO can be detachably connected to a walking boot to provide additional support and to maintain the foot, ankle and lower leg at the prescribed angle. Since the AFO can be detached from the walking boot, it may also be worn by itself during times when only support is required, such as when the patient is sleeping, resting during the day or, during later stages of recovery, it can be worn within an ordinary shoe.

A PTB can also be detachably connected or permanently attached to the walking boot. The PTB can offload some or all of a patient's weight and is advantageous over crutches, walkers or other assistive devices because it does not require weight to be borne by the upper body, which makes a PTB generally easier to use. The PTB may partially or fully suspend the patient's foot, ankle and lower leg within the walking boot.

The inventive modular design of the system disclosed herein allows the patient to remove the walking boot during periods in which it may be cumbersome or unnecessary for the patient. For example, the walking boot may be removed for sleeping or resting because it is detachable from the AFO such that the AFO can remain on the foot without the walking boot. The AFO can provide sufficient bracing and protection for activities like sleeping or resting, but without the inconvenience and discomfort caused by the added bulk and weight of a walking boot.

Advantageously, the modular design and adjustable nature of the system disclosed herein allow patients to use a single system for their complete recovery. For example, as the patient becomes able to bear more weight on the foot, the relative proportion of the patient's weight borne by the PTB and the foot bed of the boot can be appropriately adjusted to accomplish this in a controlled and systematic fashion as opposed to other methods, like crutches, which do not allow for systematic control of the weight being borne by the foot.

Further, it is advantageous that the modules of the orthopedic bracing system can be connected. For example, connecting the AFO to the walking boot prevents the AFO from sliding inside the boot, increases stability and reduces friction on the wound.

Also disclosed herein is a new design for a walking boot that is configured to work with the overall orthopedic bracing system and address long-standing problems in the art. The most widely used medical or orthopedic walking boot contains a rubber sole, metal base with foam padding and two vertically oriented metal posts with VELCRO® strips attached to the inner and outer surfaces of the posts. A soft fabric liner rests on the padded metal base. The liner attaches to the inner surface of the two metal posts with VELCRO®. The fabric liner is open in the front to accept a patient's leg. After the leg is resting in the boot, the liner is closed and 3 to 4 circumferential VELCRO® straps are attached to the outer surface of the two metal posts with VELCRO® and then tightened to hold the leg securely. There are several practical problems with this design. The circumferential VELCRO® straps adhere to themselves, adhere to one another, are detachable and frequently lost, frequently tangled, turned over, knotted and, out of anger, simply cut and discarded.

The liner suffers from similar problems. It sticks to anything with VELCRO®. It is difficult to remove from the metal posts of the boot and usually requires that all of the VELCRO® straps be taken off as well. Thus, removing the liner to wash it and place it back into the boot is frustrating in most cases. However, if the liner is not routinely washed, it becomes malodorous. Replacing the liner properly requires knowledge of the product and patience. Few patients are able replace the liner and maintain a good fit. Many patients return to the health care provider's office upset that they are unable to manage the boot with its straps and liner.

The new fabric liner disclosed herein addresses these issues. VELCRO® can be removed entirely from the side posts. In one non-limiting embodiment, the liner may be provided with two wide side pockets that slide over and capture the side posts. The opening of the pockets are along the inferior or bottom side. The side pockets are reinforced to prevent tearing. The side pockets may also be trapezoidal in configuration, e.g., wider on top and narrower on the bottom. This will allow more adjustment for different size calves. Narrower at the bottom will help to maintain proper liner and foot position with respect to the base of the boot. In general, wide side pockets allow the liner to be moved back for larger legs and forward for smaller legs.

Without VELCRO on the two side posts, the liner can be easily placed into position and removed. Removing the liner for washing, caring and reassembly is simple and easy. The liner only fits on the boot one way to prevent confusion.

Two wide straps with limited VELCRO® at the ends may be permanently attached to the liner to prevent loss of straps, poor positioning of straps, and minimize tangling of the straps. The straps may be oriented in opposite directions to facilitate tightening of the straps and secure fit of the boot. VELCRO® or other means may also be used at the bottom of the liner to fix it to the padded metal base.

Alternatively, the liner may be closed using two wide straps with loops at the ends that secure to knobs or hooks on the side posts. In this example, the side pockets may have a cutout to allow access to the knobs or hooks on the side posts. In another embodiment, the liner may be closed using a series of bands that are attached to one end of a connector with the other end of the connector being attached to the side posts. The side pockets may have a cutout to allow access to the connector on the side posts. Alternatively, the liner may be closed using a drawstring closure, side release buckles or a bail and catch system.

The liner may optionally include mesh sections to allow for increased airflow. The mesh sections may be on the back of the liner, the sides of the liner or the foot section of the liner.

The liner may also optionally include a pneumatic device for compression. The pneumatic device may be inflated using for example, an inflation bulb, a manual pump, or an open-cell foam in conjunction with an air valve.

Similar to other walking boots that may be used with the disclosed orthopedic bracing system, the AFO can be attached to the fabric liner walking boot. For example, the AFO may be attached to walking boot via a knob and rail connection, via straps that pass though the liner and the base of the walking boot and wrap around the users ankle or, in another example, a strap may connect the AFO to the liner and any of the methods described herein may be used to attach the liner to the walking boot base and posts. The AFO may also be attached to the walking boot using a continuous strap.

A PTB or a support that attaches to the patient's thigh may also be used in conjunction with the fabric liner walking boot.

A trapezoidal shaped extender pad can optionally be added to the liner for extremely large and wide legs. Currently, walking boots are fit to the size of a patient's foot but in an ever-increasing set of patients, their lower legs and calves are too large to close the liner. Frequently the boot liners are left open anteriorly or boots are oversized for the feet in order to close around the calves but this creates a tripping hazard. The extender pad can be made from the same material as the liner and easily added to the liner with VELCRO® to adequately cover larger legs.

In a first embodiment, this disclosure provides a walking boot configured to cover a portion of a user's foot, ankle and lower leg. A PTB is detachably connectable or may be permanently connected to the walking boot and configured to partially or fully suspend a portion of the user's weight. An AFO is also detachably connectable to the walking boot.

In an aspect of the first embodiment, the AFO can be configured for insertion into a shoe. For example, at a later stage of recovery the walking boot may not be required, but some bracing may still be needed for stability and to maintain the proper angle of the foot. At such a stage of recovery, the AFO may be removed from the boot and used with an ordinary shoe.

In a second aspect of the first embodiment, the AFO has a base that is configured to cover the sole and arch of the user's foot and an upright portion that is configured to cover the user's heel and the posterior portion of the user's ankle. However, one of skill in the art can appreciate that the AFO may be configured to extend up the calf or even cover the entire calf of the user.

In another aspect, the AFO can be a universal or generic fit that fits most foot types. Alternatively, the AFO may be easily molded to create a more secure fit and improved support. For example, the AFO may be molded in a healthcare professional's office.

In another aspect of this embodiment, the PTB is constructed of a posterior shell and an anterior shell and a strap connects the posterior and anterior shells. The strap may pass through a ring connected to an arcuate track in the posterior shell or the anterior shell. This provides the ability to adjust the PTB to fit users of different sizes and heights.

In another aspect, the location of the connection between the PTB and the walking boot is adjustable. Adjusting the height of the PTB further enhances the ability of the orthopedic bracing system to accommodate patients of various heights and may also improve control over the amount of weight being borne by the user's foot and ankle.

In another aspect, the PTB is configured to provide a first upward force on a user and the foot bed of the walking boot is configured to provide a second upward force on the user and the relative amounts of the first and second upward forces are adjustable.

In another aspect, a wedge is positioned under the sole of the walking boot placing the sole of the walking boot at an angle to the ground. The angle created by the wedge may be variable between 1° and 60°.

In another embodiment this disclosure provides a walking boot configured to cover a portion of the user's foot, ankle and lower leg. An AFO can be detachably connectable to the walking boot.

In one aspect, the walking boot has a foot bed and the distance between the AFO and the foot bed can be adjusted.

In another aspect, the AFO is attached to the walking boot by a track, rail, spine, extension, prominence, flush mount bracket, a snap fit connection, a hook and loop connection (e.g.,)VELCRO®), a magnet or a strap. For example, the walking boot may have a track, rail, spine, extension, prominence or flush mount bracket on an interior surface and the AFO may have a channel, slot, groove, recess, depression, receptacle, or flush mount bracket on its exterior surface configured to cooperate with the attachment mechanism on the interior surface of the walking boot. The channel, slot, groove, recess, depression, or receptacle on the exterior of the AFO would capture the track, rail, spine, extension, or prominence of the walking boot to releasably attach the AFO to the walking boot. A flush mount bracket, button, knob or lever on the exterior of the AFO may be configured to cooperate with a flush mount bracket or track in the walking boot to detachably connect the AFO to the boot. Alternatively, the AFO may have a flush mount bracket or track on an exterior surface and the walking boot may have a flush mount bracket, button, knob or lever on its interior surface. The flush mount bracket, button knob or lever on the interior of the walking boot may be configured to cooperate with the flush mount bracket or track on the AFO to detachably connect the AFO to the walking boot. Other suitable structures for attaching the AFO to the boot are also contemplated by this disclosure.

In another embodiment, a method for supporting an injured lower extremity is provided. For example, a first support member may be attached to a region surrounding the patellar tendon of the injured patient. A second support member for the area at or beneath the foot may be provided. A first upward force may be provided at the patellar region of the user by the first support member and a second upward force may be provided at the user's foot with the second support member. The amounts of the first upward force and the second upward force may be adjusted to a desired balance.

In one aspect, the magnitude of the first and second forces may be adjusted by changing the positions of the first and second support members.

In another aspect, the first support member is a PTB.

In another aspect, a prescribed angle can be maintained between the foot and the leg after the amounts of the first upward force and the second upward force are adjusted.

In another aspect, an AFO may be used to maintain the prescribed angle between the foot and the leg.

In another embodiment, a brace assembly is provided. The brace assembly includes a walking boot configured to cover at least a portion of the plantar aspect of a user's foot, and an alternative support that may be connected to the walking boot, the support having a first support portion configured to attached to the user's thigh. The alternative support may replace the PTB in certain embodiments described herein. The support includes a lockable joint. When the joint is unlocked, an angular position of the first support portion relative to the walking boot is adjustable, and when the joint is locked, the angular position of the first support portion relative to the walking boot is fixed.

In another embodiment, a brace assembly is provided. The brace assembly includes a walking boot configured to cover at least a portion of the plantar aspect of a user's foot, and an alternative support that may be connected to the walking boot, the support having a first support portion configured to attach to the user's thigh. The alternative support may replace the PTB in certain embodiments described herein. Further, the walking boot has a base axis and a calf axis and an adjustable angle β therebetween. The position of the user's calf relative to the base of the walking boot is adjustable.

In another embodiment, a brace assembly is provided. The brace assembly includes a walking boot configured to cover at least a portion of the plantar aspect of a user's foot, and an alternative support that may be connected to the walking boot, the support having a first support portion configured to attach to the user's thigh. The alternative support may replace the PTB in certain embodiments described herein. Further, the walking boot has a base whose height is adjustable.

In another embodiment, a brace assembly is provided. The brace assembly includes a walking boot configured to cover at least a portion of the plantar aspect of a user's foot, and an alternative support that may be connected to the walking boot, the support having a first support portion configured to attach to the user's thigh. The alternative support may replace the PTB in certain embodiments described herein. Further, the brace assembly includes an ankle-foot orthosis (“AFO”) detachably connectable to the walking boot.

In an aspect of the above embodiments, the alternative support further includes a second support portion configured to attach to the user's leg below the patella.

In an aspect of the above embodiments, when the joint of the alternative support is unlocked, the position of the second support portion is fixed relative to the walking boot and is adjustable relative to the first support portion.

In an aspect of the above embodiments, the first support portion may have a shape configured to surround the user's thigh.

In an aspect of the above embodiments, the walking boot may have a base axis and a calf axis and an adjustable angle β therebetween. The position of the user's calf relative to the base of the walking boot is, thus, adjustable.

In an aspect of the above embodiments, the walking boot has a base whose height may be adjustable. The height may be adjusted by a wedge or a block. The wedge or block may be attached to the foot bed of base of the walking boot. Alternatively, the wedge or block may be attached to the sole of the base of the walking boot.

In an aspect of the above embodiments, the brace assembly includes an AFO detachably connectable to the walking boot. The AFO may be attached to the walking boot by one of a rail, a track, a rail and a protrusion, a flush mount bracket, a snap fit connection, a hook and loop connection, a magnet, a strap and a continuous strap.

In an aspect of the above embodiments, the brace assembly may optionally include a pneumatic feature configured to provide compression to the user's foot, ankle and/or lower leg. The pneumatic feature may include an inflation bulb, a pump and/or a self-inflating bladder.

As noted above, one advantage of certain embodiments in accordance with this disclosure is that the patient may use a single system as disclosed herein for their entire recovery. In particular, the detachability and adjustability feature of the AFO and PTB modules in the disclosed embodiments allows the user's weight to be adjusted as the patient recovers such that additional weight may be taken from the PTB and borne by the foot/ankle.

Another advantage of certain embodiments disclosed herein is the modularity, which also makes it possible for a single system to be used for the patient's entire recovery and reduces certain perceived barriers to compliance. For example, as discussed above, when the patient is sleeping the boot may be removed and placed aside while the AFO is left on the limb such that the patient's comfort is increased due to not having to wear the bulky, heavy and ungainly boot. Similarly, the AFO can be adapted in the later stages of recovery such that the patient can slip a foot with the AFO already attached into a shoe.

Another advantage of certain embodiments disclosed herein is the adjustability of the modules in the system, which allows patients of different sizes to use the same braces. For example, the same PTB or alternative support may be adjusted to fit patients of various sizes rather than needing a PTB or support custom made for each patient. The boot, and the AFO can be designed to accommodate different size feet, ankles and lower legs.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1A is a perspective view of an orthopedic brace assembly in accordance with this disclosure with a portion of a patient's leg shown in phantom.

FIG. 1B is a side view of the orthopedic brace assembly shown in FIG. 1A.

FIG. 1C is a rear perspective view of the orthopedic brace assembly shown in FIG. 1A.

FIG. 2A is a perspective view of a PTB, a portion of which is also depicted in FIG. 1A.

FIG. 2B is a side perspective view of the PTB depicted in FIG. 2A.

FIG. 2C is a view of the PTB depicted in FIG. 2A in the open position showing the interior portions of the anterior (left) and posterior (right) shells.

FIG. 3A is a front perspective view of a walking boot as shown in the assembly depicted in FIG. 1A.

FIG. 3B is a front perspective view of a walking boot as shown in the assembly depicted in FIG. 3A with an optional cover shown.

FIG. 3C is a front perspective view of a walking boot as shown in the assembly depicted in FIG. 3A with an optional liner shown.

FIG. 4A is a front perspective view of an AFO, a portion of which is also depicted in FIG. 1A.

FIG. 4B is a side view of the AFO shown in FIG. 4A.

FIG. 4C is a rear view of the AFO shown in FIG. 4A.

FIG. 5 is a view of the orthopedic brace assembly shown in FIG. 1A partially disassembled.

FIG. 6 is another perspective view of the orthopedic brace assembly shown in FIG. 1A with a portion of a patient's leg, foot and ankle show in phantom.

FIG. 7A depicts a system in accordance with this disclosure with a simple force diagram.

FIG. 7B depicts a portion of the system shown in FIG. 7A with further illustration of the forces.

FIG. 7C shows a fragmentary sectional view of a portion of the system shown in FIG. 7A.

FIG. 7D depicts a side view of an orthopedic brace assembly in accordance with this disclosure.

FIG. 8A depicts a front perspective view of an orthopedic brace assembly in accordance with this disclosure.

FIG. 8B depicts a simple force diagram from the orthopedic brace assembly shown in FIG. 8A.

FIG. 8C depicts a simple force diagram from the orthopedic brace assembly shown in FIG. 8A.

FIG. 8D is a view of an orthopedic brace assembly according to FIG. 8A partially disassembled.

FIG. 9A is a perspective view of a frame of a walking boot for use in an orthopedic brace assembly according to this disclosure.

FIG. 9B is a front perspective view of the frame of a walking boot shown in FIG. 9A.

FIG. 9C is a perspective view of the frame of the walking boot shown in FIG. 9A showing a liner partially attached.

FIG. 9D is a perspective view of the frame of the walking boot shown in FIG. 9A showing a liner partially attached.

FIG. 9E shows an AFO that can be used with a walking boot in accordance with this disclosure.

FIG. 9F is a front perspective view of the walking boot shown in FIG. 9A showing the liner attached.

FIG. 9G is a front perspective view of the walking boot of FIG. 9A showing the liner and the AFO attached.

FIGS. 10A, 10B, 10C, 10D, 10E, 10F, 10G, 10H and 10I are illustrations of walking boots with various attachment configurations for the liner.

FIGS. 11A, 11B, 11C, 11D, 11E, 11F, 11G, 11H, 11I, 11J, 11K, 11L, 11M and 11N show various options for attaching an AFO to a walking boot.

FIGS. 12A, 12B, 12C, 12D, 12E, 12F, 12G, 12H and 12I illustrate different configurations of pneumatic features for providing compression in a walking boot of this disclosure.

FIGS. 13A, 13B and 13C show an alternate embodiment of an orthopedic bracing assembly including a walking boot having a support that attaches to the user's thigh and upper calf area.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

In this disclosure, terms such as “horizontal” and “vertical” are generally used to establish positions of individual components relative to one another rather than an absolute angular position in space. Further, regardless of the reference frame, in this disclosure terms such as “vertical,” “parallel,” “horizontal,” “right angle,” “rectangular” and the like are not used to connote exact mathematical orientations or geometries, unless explicitly stated, but are instead used as terms of approximation. With this understanding, the term “vertical,” for example, certainly includes a structure that is positioned exactly 90 degrees from horizontal, but should generally be understood as meaning generally positioned up and down rather than side to side. Other terms used herein to connote orientation, position or shape should be similarly interpreted. Further, it should be understood that various structural terms used throughout this disclosure and claims should not receive a singular interpretation unless it is made explicit herein.

Further, it should be understood that all terms used throughout this disclosure and claims, regardless of whether said terms are preceded by the phrases “one or more, “at least one, or the like, should not receive a singular interpretation unless it is made explicit herein. That is, all terms used in this disclosure and claims should generally be interpreted to mean “one or more” or “at least one.”

The terms “support” and “alternative support” are used interchangeably throughout this disclosure. Minimally, these terms refer to a brace or support including first and second support portions. The first support portion may be configured to surround the user's thigh and the second support portion may be configured to surround the patient's leg at or below the patella.

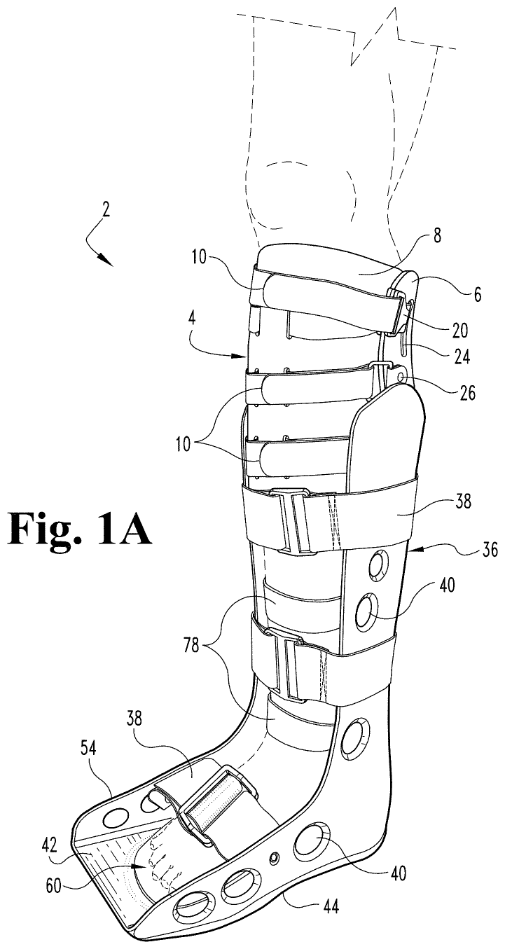

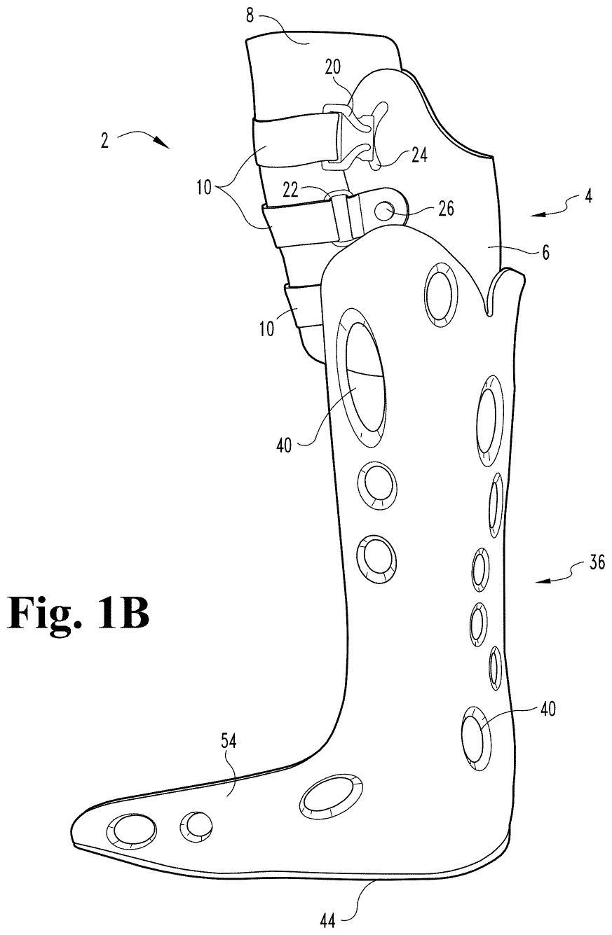

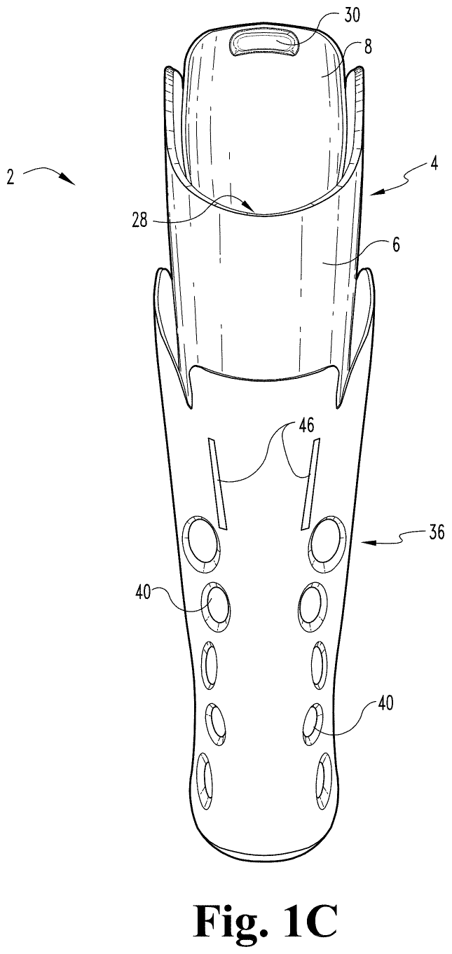

FIGS. 1A-1C show an embodiment of an orthopedic brace assembly 2. The configuration shown includes a patellar tendon bearing brace (“PTB”) 4, which is attached to a walking boot 36. The patient's foot is inside an ankle-foot orthosis (“AFO”) 60, which is removably attached to the walking boot 36. In this configuration, the AFO 60 may be fully suspended within the walking boot 36 such that the AFO 60 does not contact the base of the walking boot 36. Alternatively, in this configuration, the AFO 60 may also contact the base of the walking boot 36. As can be appreciated, different configurations are possible such as, for example, a PTB 4 attached to a walking boot 36 without an AFO 60. In another possible configuration, an AFO 60 may be attached to a walking boot 36 without a PTB 4 and the AFO 60 may be in full contact with the base of the walking boot.

### PTB

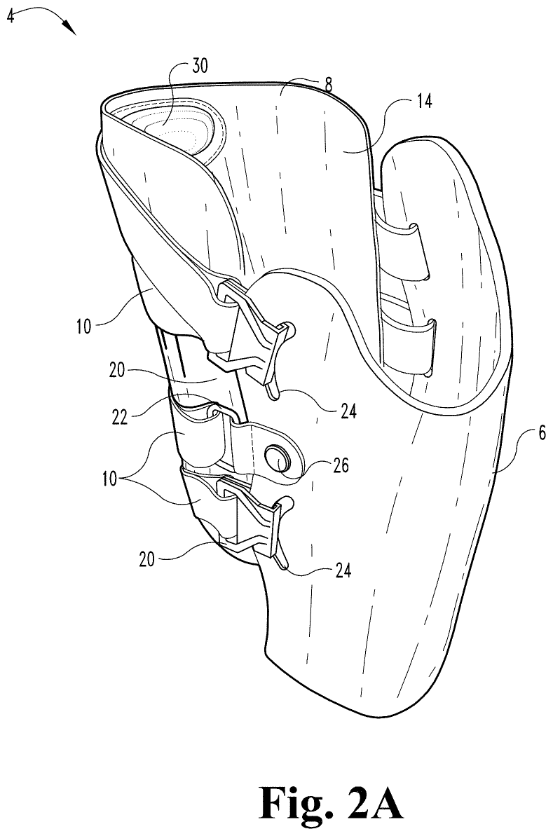

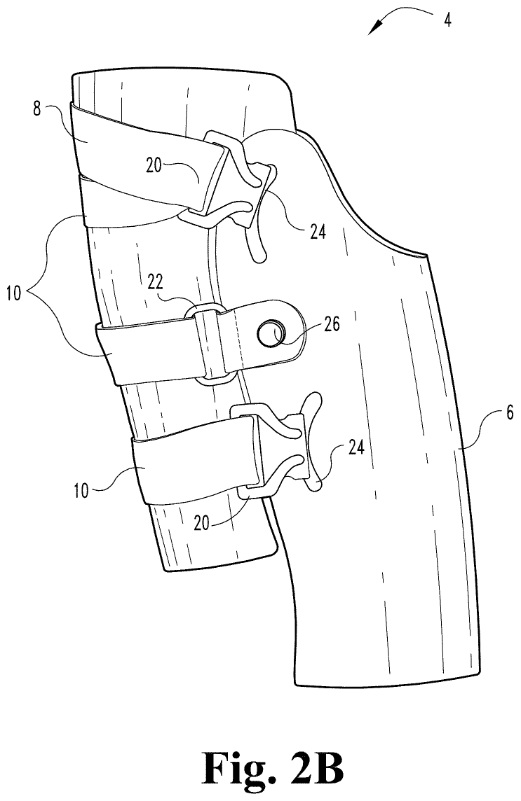

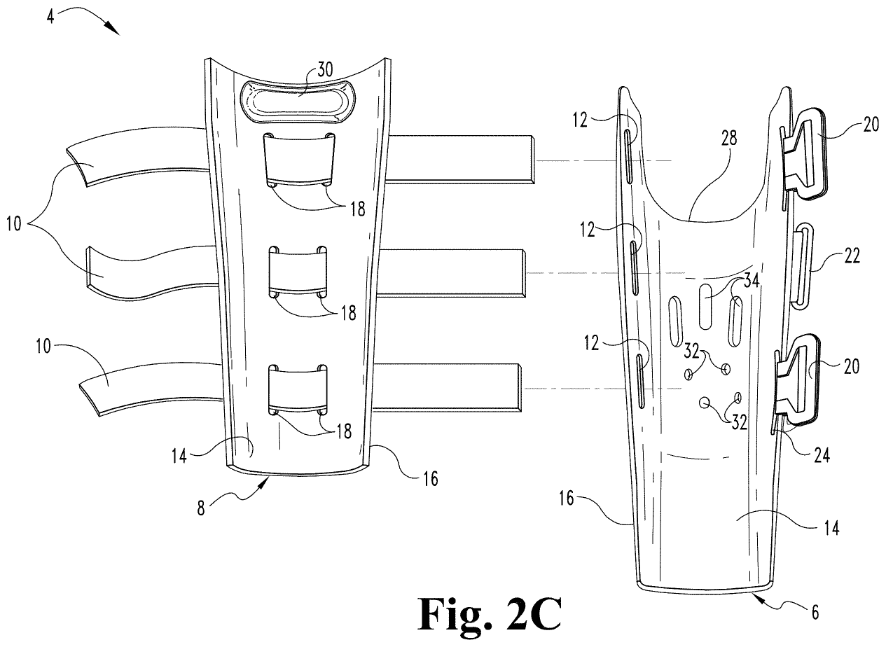

The first module of the orthopedic bracing system 2 is the PTB 4. FIGS. 2A-2C depict an example of a PTB 4.

The PTB 4 may be formed in two parts, the posterior shell 6 and the anterior shell 8. The posterior shell 6 and the anterior shell 8 may be connected by straps 10. Straps 10 are formed from hook and loop material (e.g., VELCRO®) in the embodiment shown. However, one of skill in the art will appreciate that the straps 10 could be formed from a variety of materials including leather, metal, an elastomeric material, plastic or some combination of materials. One end of the straps 10 is anchored on one side of the posterior shell 6 at fixed slots 12. The free end of each strap 10 is then stretched around the anterior shell 8 to a pair of strap guides 18. The free end of the strap 10 is passed through the pair of strap guides 18 and stretched around the remaining portion of the anterior shell 8. The free end of strap 10 passes through either an adjustable ring 20 or a buckle 22, shown in FIG. 2C as a rectangular ring, that is attached to the posterior shell 6. Finally, the free end of strap 10 is secured to itself using the hook and loop fastener. It can be appreciated that the free end of strap 10 could be secured differently, for example, with snaps, buttons, a latching mechanism, locking mechanism, buckle, ratchet system, small cables, etc.

Other configurations of PTB 4 will also work with this disclosure. For example, the posterior shell 6 and the anterior shell 8 may be connected by a wide variety of structures instead of straps 10, such as hinges, bands, latching mechanisms, interlocking tracks, locking mechanisms, buckles, ratchet systems, small cables, etc. The connection between posterior shell 6 and anterior shell 8 may also include bail and catch or ratcheting buckles to create a tighter fit. In another alternative, the posterior shell 6 and the anterior shell 8 may be integrally formed and there may be a slit in the PTB 4 to allow the leg to pass through before the edges of the slit are drawn together with laces or a zipper.

As can be seen in FIG. 2B, adjustable rings 20 are mounted on an arcuate track 24 in the posterior shell 6. The adjustable rings 20 may be slid up or down the arcuate tracks 24 to obtain different heights and angles for the position of the adjustable ring 20. This allows the PTB 4 to be adjusted to fit patients of different heights, weights and leg/calf diameters. The buckle 22 may be mounted to posterior shell 6 by, for example, the rivet 26 as shown or by welding, stitching, adhesives, or by any method known in the art.

The PTB 4 is held in place on the leg by tightening straps 10 such that the patellar bar 30 (also referred to as a “patellar tendon knob”) in the anterior shell 8 is pressed against the patient's patellar tendon, inferior surface of the patella and tightened to the calf. The patellar bar 30 is a protrusion in the anterior shell 8 of PTB 4. It is contemplated that PTB 4 may be held in place by other or additional structures such as a compressible foam liner or air bladders.

The exterior of PTB 4 may be formed from a rigid or semi-rigid material such as plastic, plasticized organic fabrics, etc. To improve user comfort, the interior surface 14 of the PTB 4 may be formed from a nonslip material to prevent rubbing and irritation to the skin and also a softer material, such as foam, fabric or a combination of softer materials. Vent holes 34 may be provided in the posterior shell 6. Further, the top edge of posterior shell 6 may be an arc 28 that dips below the back of the knee to avoid pinching the back of the leg when the user sits.

### Walking Boot

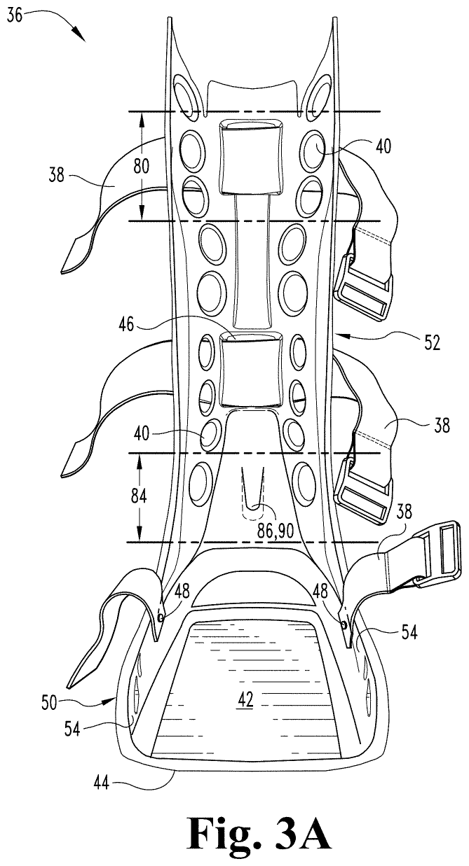

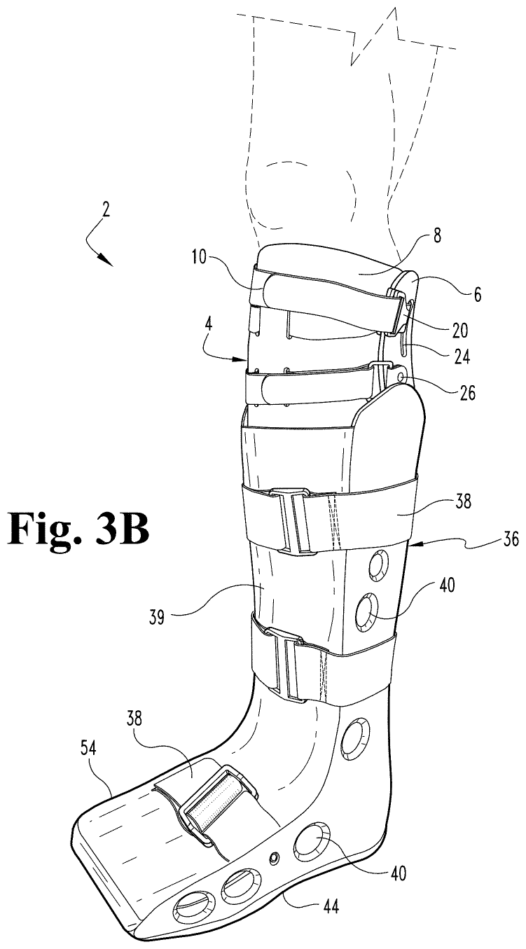

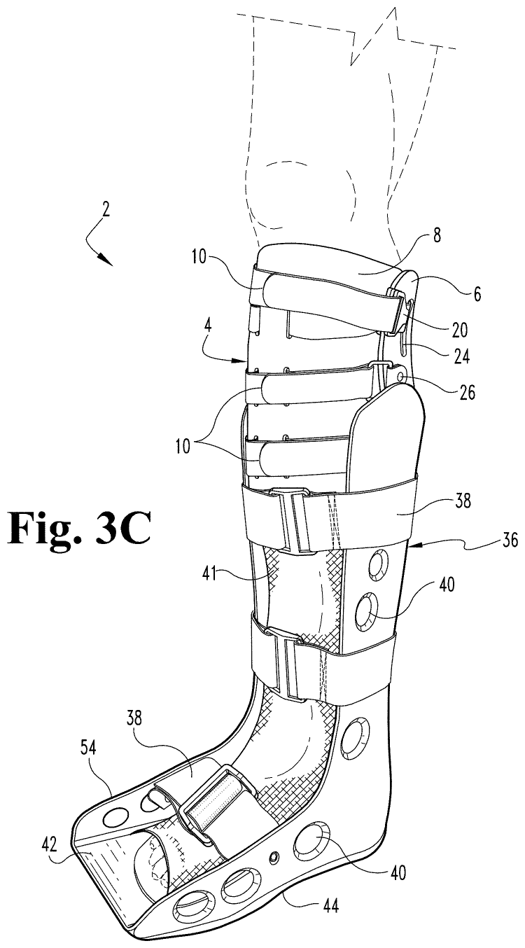

The second module of the orthopedic bracing system 4 is the walking boot 36, depicted in FIGS. 3A-3C.

The walking boot 36 includes a base 50 and an upper section 52. The base 50 and the upper section 52 may be integrally formed from plastic or other suitably rigid, lightweight materials. For example, the walking boot 36 may be formed from any rigid, hard durable material including plastic, metal, steel, aluminum, fiberglass, carbon fiber, composite material or any combination thereof. In another, non-limiting example, the walking boot 36 may also be formed from, e.g., polypropylene or polyethylene.

Alternatively, the upper section 52 of walking boot 36 may be formed by connecting rigid uprights to base 50. The uprights can be spaced apart from each other. For example, there may be one upright may be positioned near the heel of the user and additional uprights may be located on the lateral sides of the user's leg. The uprights may be formed from any rigid material, e.g., plastic or metal, e.g. steel. For example, the uprights may be formed from any rigid, hard durable material including plastic, metal, steel, aluminum, fiberglass, carbon fiber, composite material or any combination thereof. The uprights may also be formed from, e.g., polypropylene or polyethylene. Fabric may be stretched around the uprights to form an enclosure for the user's leg.

The base 50 of the walking boot 36 has a sole 44 made from elastomeric or rubber material with treads for better traction. The top surface of the base 50 is a broad, flat foot bed 42 that can accommodate a range of foot sizes. The foot bed 42 may be surrounded or partially surrounded by a sidewall 54 that extends vertically from the foot bed 42 to protect the sides of the foot. Optionally, the base 50 of the walking boot 36 may include straps 38 attached to sidewalls 54 by, e.g., rivets 48 as shown in FIG. 3A or by some other suitable attachment mechanism such as adhesive, glue, welding, screws, bolts, thread, etc., or the straps 38 may be removably attached to sidewalls 54 by way of, e.g., a snap, button, or latching mechanism. The straps 38 may be used to secure the base 50 of the walking boot 36 to the patient's foot during some stages of recovery. Alternately, as shown in FIG. 3B, the straps 38 may be used to secure a front section or a cover 39 of the walking boot 36 that covers the anterior ankle and leg and the dorsum of the foot.

The upper section 52 of the walking boot 36 extends from the base 50 upward to approximately the proximal end of the patient's calf. It surrounds the posterior side and lateral sides of the patient's lower leg and is open on the anterior side. The upper section 52 of walking boot 36 may be secured to the patient's leg with, e.g., hook and loop straps 38. However, other attachment mechanisms are also contemplated such as snaps, buttons, hooks, latching mechanisms, buckles, ratchet mechanisms, small cables, etc. The straps 38 may be attached to the walking boot 36 by the same mechanisms discussed in relation to the straps 38 for base 50, or they may be attached using strap guides 46 as shown in FIG. 3A.

The walking boot 36 may include optional features for patient comfort, such as vent holes 40. It may also include a cover 39, as shown in FIG. 3B, or a liner 41, as shown in FIG. 3C.

### AFO

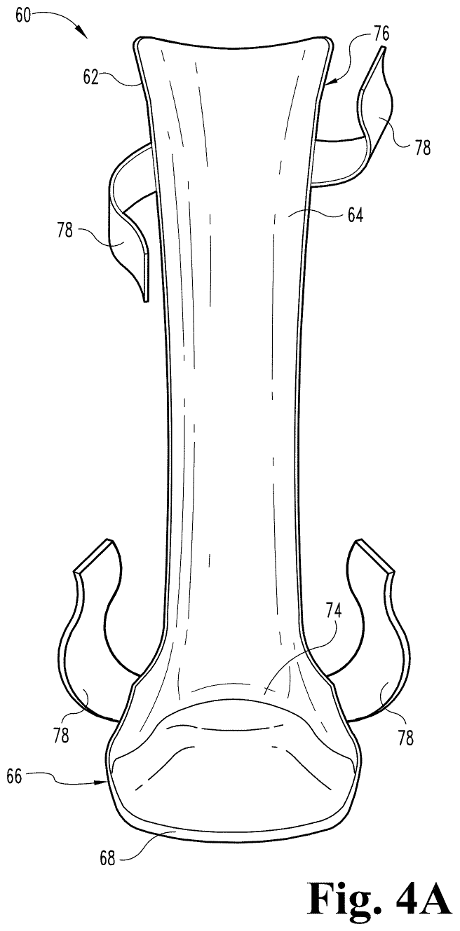

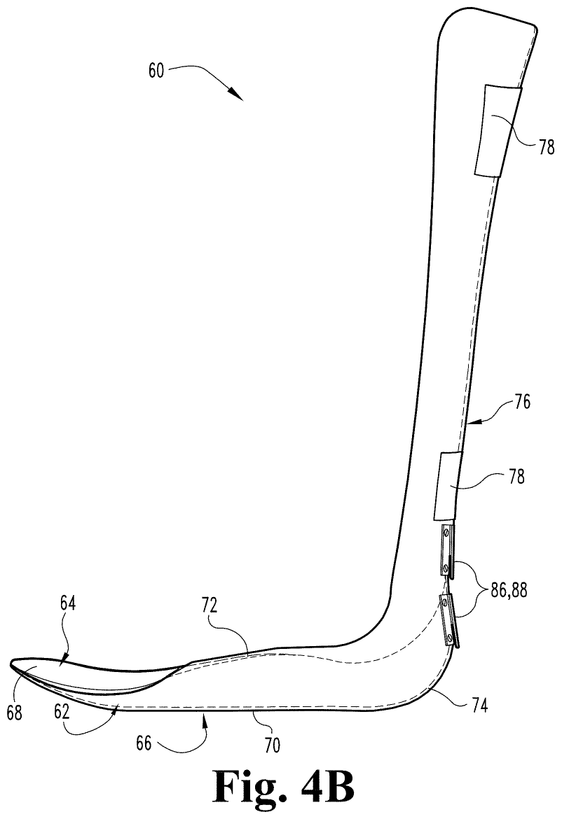

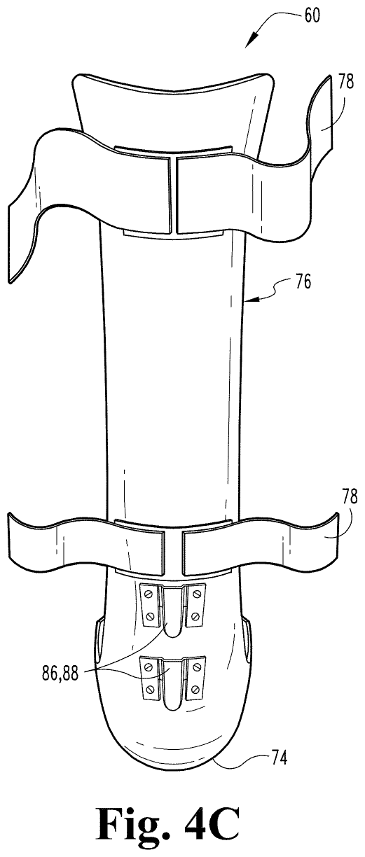

The AFO 60 is the third module of the bracing system, depicted in FIGS. 4A-C. The AFO 60 maintains the foot and ankle at a prescribed angle. For example, when the foot and ankle are suspended by the PTB, the AFO can prevent or minimize the natural plantar flexion that occurs when the foot is unweighted or hanging free. The AFO can also prevent or minimize the degree of dorsiflexion when the user walks on the foot.

The exterior shell 62 of AFO 60 is made from a material such as a rigid or semi-rigid plastic, such as for example, polypropylene, copolymers, polyethylene or any suitable plastic, that provides structure for stabilizing the foot and ankle. The interior of the AFO 64 can be made of a softer material for comfort such as fabric or foam, for example, the product sold under the trademark PLASTAZOTE®, manufactured by Zotefoams.

The base 66 of the AFO 60 is intended to accommodate a range of sizes. It may angle upward slightly at the toe 68. The arch 70 is shown flat such that the AFO 60 may be worn on either the right or left foot. It is also contemplated that the base 66 may include a curved arch 70 to provide additional support. A sidewall 72 extends upward from the arch 70 to the heel 74 to protect the instep of the foot. The heel 74 of the base is semi-spherical to accommodate the patient's heel.

The upright portion 76 of the AFO 60 extends upward from the heel 74. The upright portion 76 is curved to fit around the posterior portion of the lower leg. In the embodiment shown, the anterior side of the leg is exposed.

The AFO 60 may be secured to the patient's leg and foot using straps 78. The straps 78 may, by way of non-limiting example, be fastened with hook and loop, snaps, buttons, an elastomeric material, or any suitable material known in the art. The AFO 60 may also be secured to the foot and/or leg by some other device such as a bandage, a wrap, a sock or some combination of methods.

The AFO 60 may be a generic or universal fit that accommodates most foot sizes and shapes. It may also be easily molded, for example, in a healthcare professional's office, to provide a more secure fit without the need for an orthoptist or measuring. The AFO 60 may also be custom-made for the user. The AFO 60 may be reversibly attached to the walking boot 36, may rest on the footbed 42 of the walking boot 36 or may be suspended in the walking boot 36 with or without the use of a PTB 4.

### Orthopedic Bracing System

The modules of the orthopedic bracing system 2 are designed to work in combination to provide the required level of stabilization, protection and offloading of the patient's weight.

In particular, as shown in FIG. 5, the posterior shell 6 of the PTB 4 can be removably and adjustably attached to the upper section 52 of the walking boot 36 in the attachment region 80 (shown in FIG. 3). The attachment mechanism 82 may be a low profile/flush mount locking mechanism, bolts, screws, interlocking tracks or any fastener suitable for creating a releasable attachment.

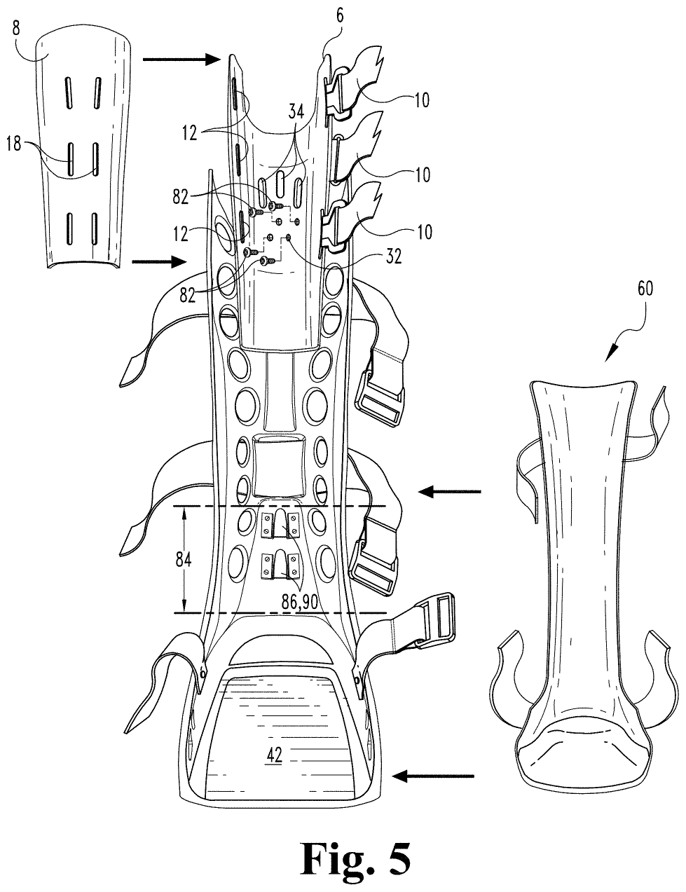

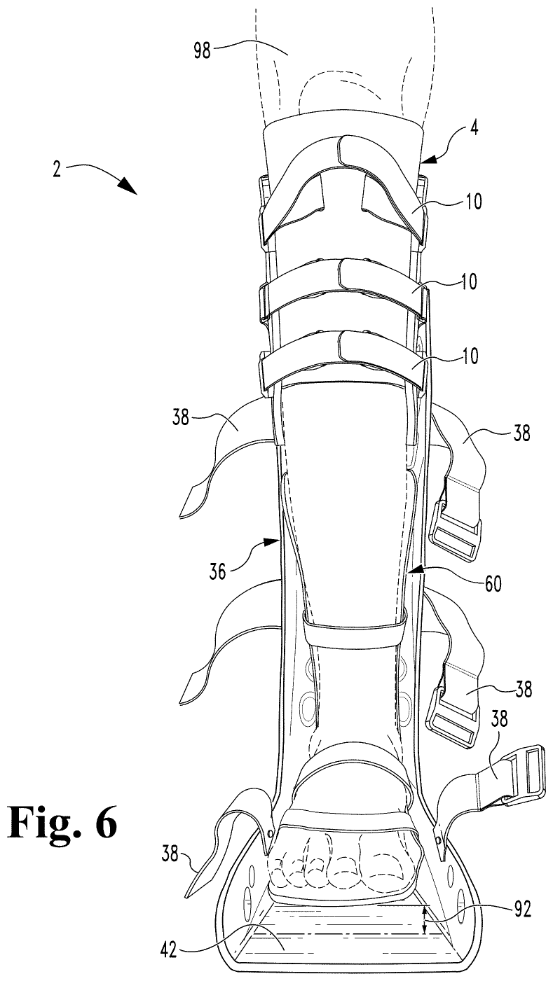

As can be seen in FIG. 6, when the PTB 4 is attached to the walking boot 36, it may suspend the user's foot above the foot bed 42 of the walking boot 36 creating a gap 92. In this arrangement, no weight is borne by the user's foot and ankle. The AFO 60 may be used to support the foot and prevent plantar flexion.

It is further contemplated that the attachment mechanism 82, which attaches PTB 4 to walking boot 36, may be adjustable to allow the percentage of the patient's weight being borne by the user's foot and ankle to range from approximately 0%, as shown in FIG. 6, to approximately 100%. For example, the walking boot 36 may have multiple sets of bolt holes 32 at higher and lower points in attachment region 80 and the PTB 4 may be attached to walking boot 36 using the bolt holes 32 at the desired height to control the degree of contact between the patient's foot and the foot bed 42 of the walking boot 36. Alternatively, the PTB 4 may be attached to a track that allows the PTB 4 to slide up and down the walking boot 36. The track may further include a locking mechanism to hold the PTB 4 at the desired height. In another alternative, the walking boot 36 may have strap guides at various heights and the PTB 4 may be attached by straps at the desired height. In still another example, the walking boot 36 may have a moving platform, which can be adjusted by a lever or a knob and the PTB 4 may be attached to the platform.

The foot bed 42 may also be adjustable. For example, it may be raised or lowered using a knob, or it might include a bladder than can be inflated or deflated. In another example, it may be configured to accommodate attachments, such as inserts, that would effectively raise the level of the foot bed 42.

In the same connection, the degree of hydrostatic compression created by the PTB 4 may also be adjustable. For example, the straps 10 of PTB 4 may be configured to apply a greater or lesser force to the user's leg. Alternatively, the PTB 4 may have an internal air bladder that may be inflated or deflated to adjust the compressive force of PTB 4 on the user's leg. In another alternative, a ratcheting buckle may be used to adjust the force.

When no longer needed, the PTB 4 can be removed from the walking boot 36. The user may continue to use the other modules of the orthopedic bracing system. For example, the AFO 60 may be used in combination with the walking boot 36 or the AFO 60 may be used with an ordinary shoe.

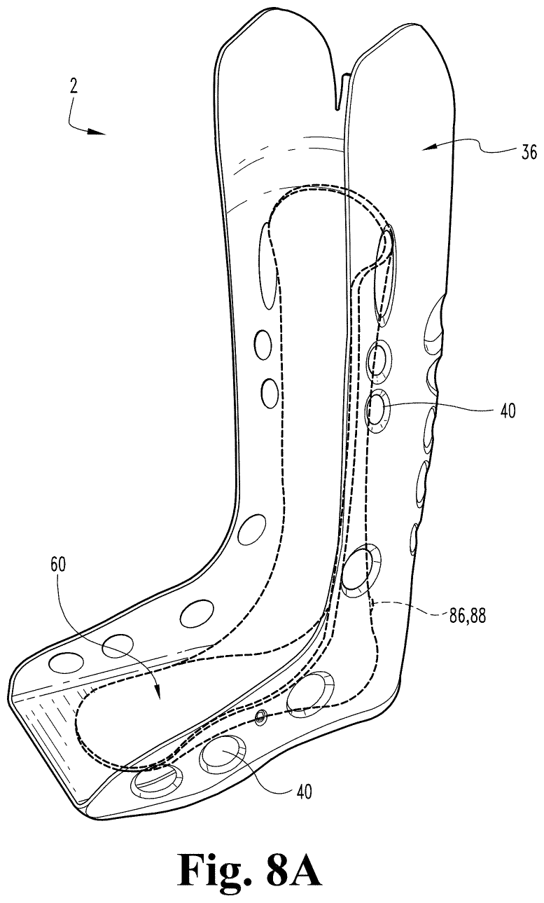

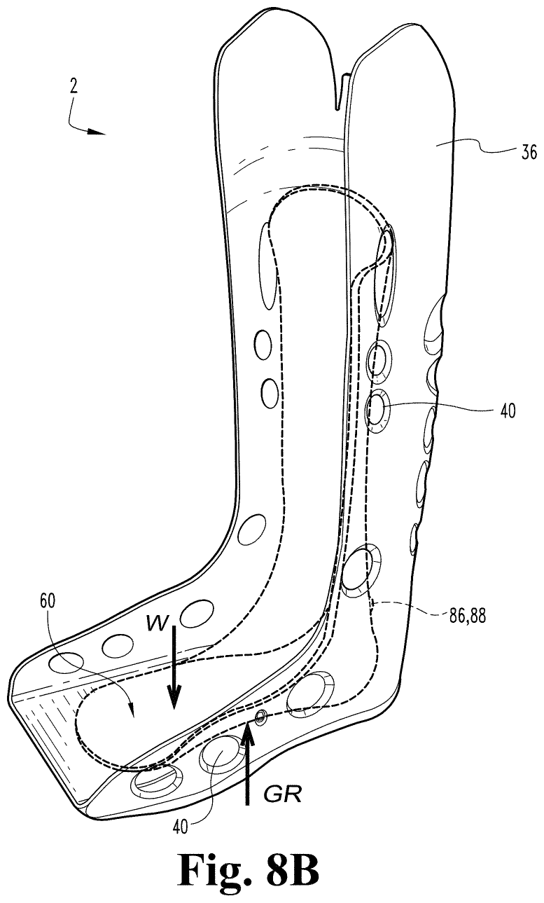

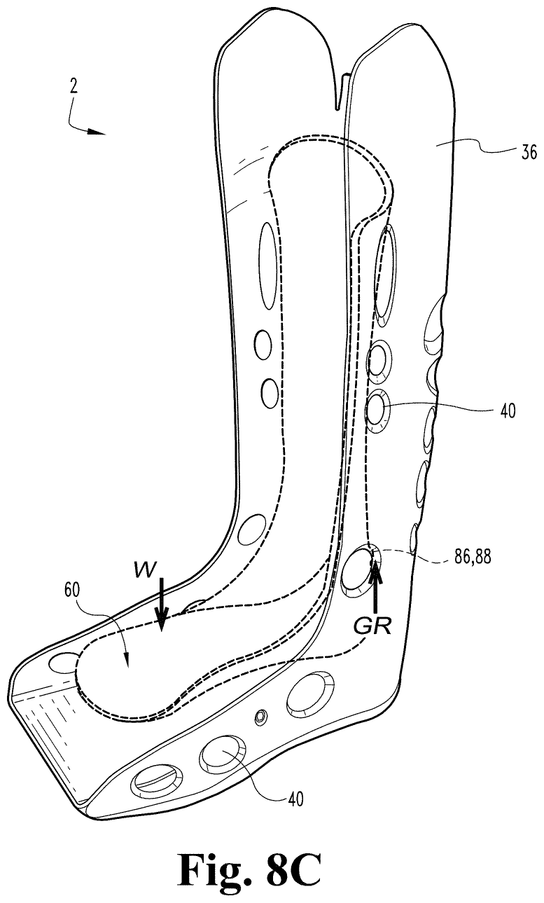

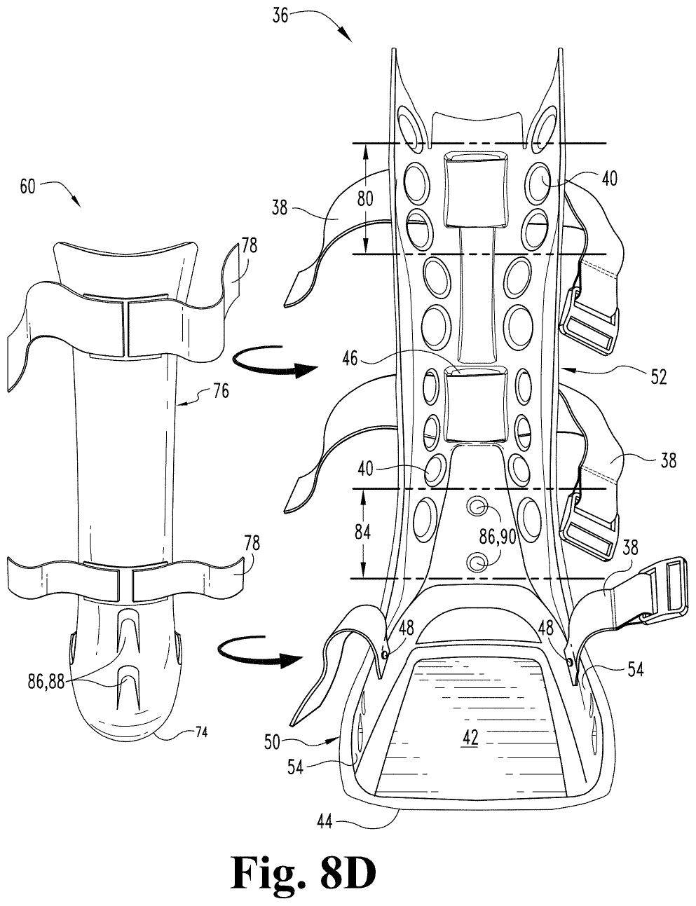

The AFO 60 may be configured to be releasably attached to the upper section 52 of the walking boot 36 at attachment region 84, as shown in a non-limiting embodiment in FIG. 5. The attachment region 84 may be larger or smaller than it is depicted in FIG. 5 or FIG. 8D. In one non-limiting embodiment, the attachment mechanism 86 may be a flush mount bracket with a clip 88 on the back of the AFO 60, shown in FIG. 4C that is configured to connect with a clip 90 in attachment region 84 of the walking boot 36, shown in FIG. 5. The attachment mechanism 86 may also be any suitable structure known in the art including, e.g., a snap fit connection, straps, a knob configured to connect with a track, hook and loop material (e.g., VELCRO®) or the like. Alternatively, as shown in FIG. 8D, the AFO 60 may have a cut out track 86, 88 in the exterior of the upright portion 76 that may cooperate with a knob or post 86, 90 in the walking boot 36 to form the detachable connection. Other embodiments are also contemplated. For example, the AFO 60 may be attached to the foot bed 42 of walking boot 36 by various means such as hook and loop material, latches, magnets, snap fit connection or any other suitable means.

The attachment between the AFO 60 and the walking boot 36 advantageously prevents movement of the AFO 60. This improves stability and reduces the risk of friction wounds.

When the walking boot 36 is used in combination with the PTB 4, as in FIG. 6, the AFO 60 can maintain the patient's ankle and foot in a prescribed position to avoid, for example, contracture. This is particularly important when a high percentage of the patient's weight is being offloaded to the PTB 4 such that the patient's foot may be completely suspended above the foot bed 42 of the walking boot 36.

Alternatively, in a configuration of the orthopedic bracing system 2 that does not include a PTB 4, the AFO 60 may rest directly on the foot bed 42 of the walking boot 36 as shown in FIG. 8A. In such a configuration, the connection between the AFO and the walking boot may be made on the foot bed 42, rather than the upper section 52. The AFO 60 may also be attached to the walking boot 36 above the foot bed 42 such that the AFO is suspended in walking boot 42, as shown in FIG. 8C. For example, the AFO 60 may be slidably mounted to a track or rail in the walking boot 36, which may serve as a guide while preventing weight bearing.

Since the connection between the AFO 60 and the walking boot 36 is releasable, the patient can remove the walking boot, for example, when they are sleeping, sitting down or resting. The AFO 60 will continue to provide stability and proper positioning of the foot, just with a lower degree of protection from external forces, which is appropriate for activities such as sleeping or relaxing.

When the patient's recovery has progressed to the point where the walking boot 36 is no longer required, the AFO 60 may be removed from the walking boot 36 and used with an ordinary shoe. The AFO 60 can continue to provide stability and positioning for the foot and ankle, but without the extra structure of a full walking boot.

### Method of Using the Orthopedic Bracing System

The particular structure and features of the brace assembly disclosed above can be better understood with regard to the following description of how the assembly is fitted onto a patient's foot/ankle and then adjusted to proportion the weight as desired.

The orthopedic bracing assembly 2 is donned by first attaching the AFO to the injured foot or ankle. The PTB 4, if needed, can be attached to the walking boot 36 and adjusted to a distance from foot bed 42 that can accommodate the user's height and can achieve the desired proportioning of weight bearing between the patellar region and the foot. It is possible, also, to adjust foot bed 42 to create the desired distance between PTB 4 and foot bed 42. For example, the foot bed 42 may be raised by attaching an insert on top of the foot bed 42. The foot bed 42 may also be raised using an inflatable bladder. In another alternative example, the foot bed 42 may be a platform that can be raised and lowered by turning a knob. The user's leg may be placed into the walking boot 36 and the AFO may then be attached to the walking boot 36. The anterior shell 8 of the PTB 4 may be adjusted such that the patellar bar 30 is in contact with the user's patellar tendon and the straps of the PTB 4 can be tightened. Finally, walking boot 36 may be secured to the leg.

A method for supporting an injured lower extremity can be provided using the disclosed orthopedic bracing system. In one embodiment of such a method, a first support member is attached to a region surrounding the patellar tendon and calf of the patient and a second support member is provided for an area at or beneath the sole of the foot of the injured leg. The first support member may, for example, be a PTB 4. A first upward force is provided on the patellar region and calf by the first support member. A second upward force is provided at the foot by the second support member. The second support member may, for example, be the foot bed 42 of the walking boot 36. The amounts of the first upward force and the second upward force can then be adjusted to a desired balance.

The adjustment in force may be made by changing the positions of the first and second support members. In particular, the adjustment may be made by changing the position of the attachment of the support members to an orthopedic walking boot.

In a further aspect of the method, a prescribed angle between the foot and leg may be maintained after the amounts of the first upward force and the second upward force are adjusted. The prescribed angle between the foot and leg can be maintained using an AFO 60. For example, when the first upward force makes up all or nearly all of the upward force, an AFO 60 can maintain the foot at the prescribed angle, rather than letting it dangle from the ankle.

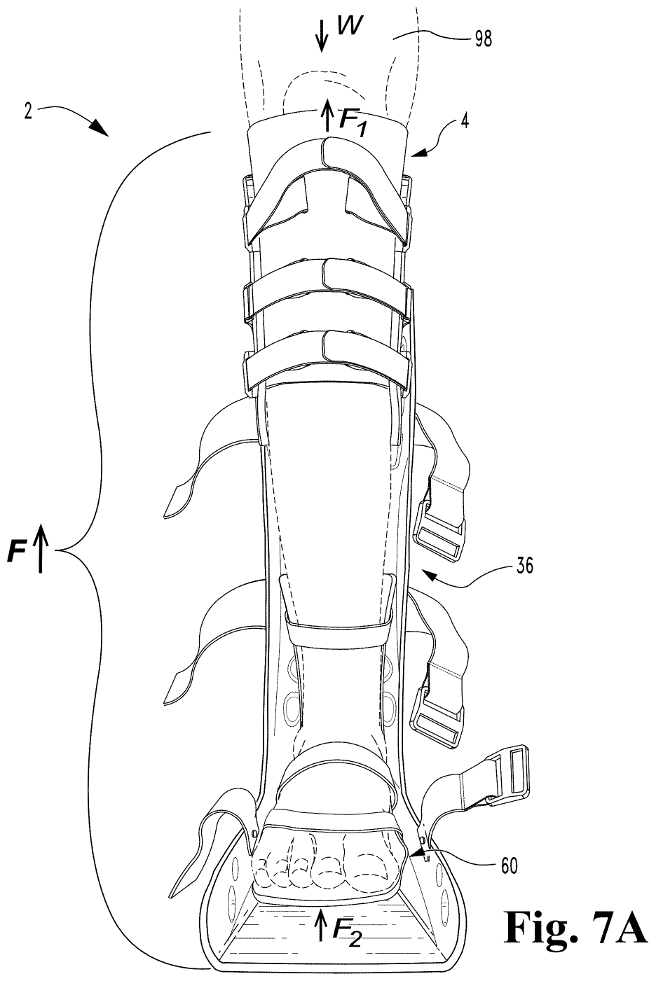

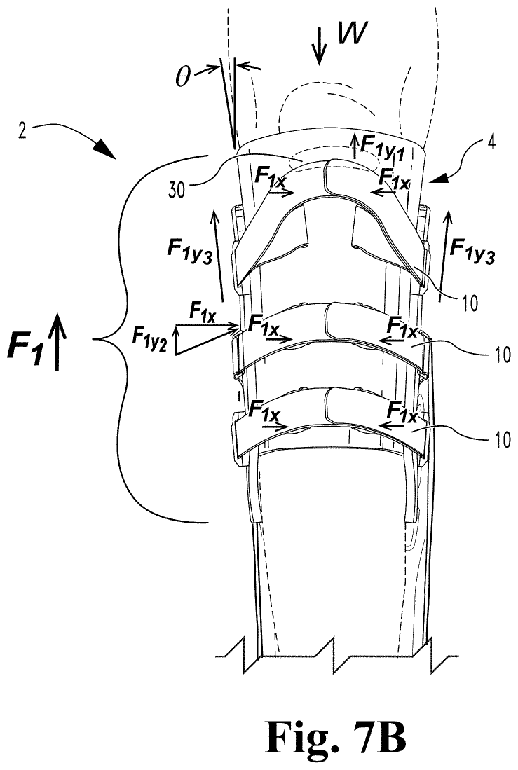

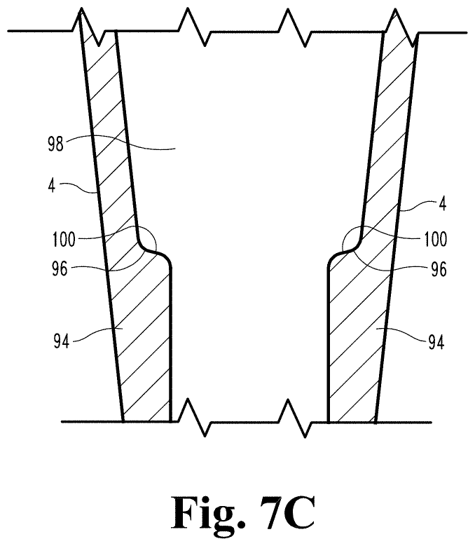

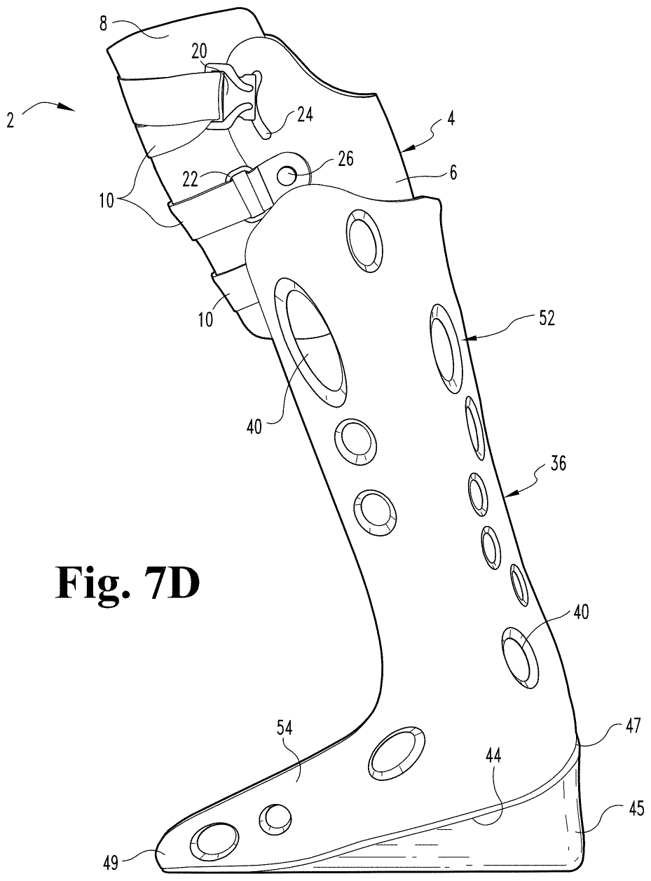

FIG. 7A shows a simple force diagram. In FIG. 7A, the entire orthopedic bracing system 2 provides an upward force F on the leg of the user that is equal to the force from the user's weight, shown as vector W.

In an exemplary method for supporting an injured lower extremity, a first support member is attached to a region surrounding the patellar tendon and calf of the injured patient and a second support member is provided for an area at or beneath the foot of the injured leg. In the example shown in FIG. 7A, the first support member is a PTB 4 and the second support member is foot bed 42 of the walking boot 36.

The upward force F is divided into two parts. A first upward force F1 can be applied in the area of the calf, the proximal tibial plateau and the patellar tendon with the first support member and a second upward force F2 can be applied to the foot by the second support member. The sum F of the first upward force F1 and the second upward force F2 will equal the downward force W from the weight of the user as the user walks or stands. Of course, the force W will vary during movement of the user such as walking and may at times exceed the weight of the user.

In the example shown in FIG. 7A, the PTB 4 and the AFO 60 are attached to the walking boot 36 at their highest positions and the PTB 4 is adjusted to its tightest position. In this configuration nearly all of the weight of the user is borne by the PTB 4 and nearly none of the weight of the user is borne by the foot or ankle of the user. Thus, F≈F1 and F2≈0.

The proportion of F1 and F2 to the total force F can be adjusted to a desired balance. For example, F1 may be from about 0% to about 100% of F and F2 may be from about 0% to about 100% of F. Within these ranges, F1 may be 100% of F, from 95-100% of F, from 90-95% of F, from 80-90% of F, from 70-80% of F, from 60-70% of F, from 50-60% of F, from 40-50% of F, from 30-40% of F, from 20-30% of F, from 10-20% of F, from 0-10% of F. F2 may be 0% of F, from 0-5% of F, from 5-10% of F, from 10-20% of F, from 20-30% of F, from 30-40% of F, from 40-50% of F, from 50-60% of F, from 60-70% of F, from 70-80% of F, from 80-90% of F, from 90-100% of F or 100% of F.

The proportion of F₁ and F₂ can be adjusted by changing the positions of the first and second support members. For example, as described above, PTB 4 can be attached at a lower position on walking boot 36 such that AFO 60 is in contact with the foot bed 42 of walking boot 36. Alternatively, as also discussed above, foot bed 42 of walking boot 36 may be raised such that it is in contact with AFO 60. The upward force F₂ is primarily a function of how firmly the foot and AFO 60 contact the foot bed 42.

Unlike the foot bed 42, which provides a substantially horizontal surface that readily provides an upward force F2, the PTB may rely on a variety of features to provide the force F₁. For example, as shown in FIG. 7B, the PTB 4 has a conical or “frustoconical” shape, in that the top end of PTB 4 has somewhat larger diameter than the bottom end. As such, by applying radially inward forces F₁_{ₓ} onto the patellar and calf region by virtue of tightening the straps 10, a slightly angled surface with respect to vertical is provided by the PTB, which in turn provides the vertical force vector shown as F₁_{_{y2}} in FIG. 7B. Similarly, another somewhat horizontal surface upon which the tendon can rest is provided by patellar bar 30, which provides the upward force F₁_{_{y1}}. Of course, as the straps 10 are tightened the static frictional force between the patient's leg and the inner surface of PTB 4 provides another component of the vertical upward force F₁_{_{y3}}. One of skill in the art would appreciate that there may be other structural features to provide vertical upward forces from the PTB 4 that contribute to the total upward force F₁ applied by the PTB 4.

Another example of a structural feature that can contribute to the upward force F₁ applied by the PTB 4 is shown in FIG. 7C, which shows a fragmentary sectional view of a portion of PTB 4 having an inflatable bladder portion 94 that, when inflated, forms a slanted somewhat horizontal surface 96. As the bladder is inflated in a predetermined manner, the patient's calf 98 is compressed such that the soft tissue conforms to the shape of the bladder and thereby provides a complementary slanted horizontal surface 100, which abuts against surface 96. In this manner, the corresponding horizontal surfaces of the calf and anatomical features of the patient can be enhanced to better provide the necessary vertical force component. In another, embodiment, discussed below, the PTB 4 may be replaced with an alternative brace that extends to the user's thigh and may be adjusted to allow force transmission to the back of the thigh as well as the knee. From these teachings, one of skill in the art would readily recognize other variations of PTB 4 to create and enhance the vertical force needed to support the force of the patient's weight W.

Another example of a structural feature that can contribute to the upward force F₁ applied by the PTB 4 is shown in FIG. 7D. A wedge 45 may be inserted under the sole 44 of walking boot 36, causing the heel 47 of walking boot 36 to be elevated. When the heel 47 of the walking boot 36 is elevated above the toe 49, the upper section 52 of walking boot 36 and the PTB 4 are placed at less than a 90° angle to the ground. This provides additional horizontal surface area that can be used to increase F₁. The wedge 45 may be used to place the sole 44 at an angle to the ground of various and adjustable degrees throughout the patient's recovery. In some embodiments, the wedge 45 may create angles of 0°-85°, inclusive, between the sole 44 and the ground.

If an AFO 60 is used in connection with certain embodiments, such as FIG. 7D, the attachment mechanism 86 between the AFO 60 and the walking boot 36 may preferably include a sliding mechanism, such as interlocking tracks or a knob and track system. The base 66 of the AFO 60 may then move relative to the upper section 52 of the walking boot 36. For example, the base 66 of AFO 60 may be able to move from 0 and 2 inches, from 0-1 inch, from 0-0.5 inches, from 0-0.25 inches with respect to upper section 52 of the walking boot 36.

Several possible alternatives for adjusting F₁ and F₂ are disclosed above. The ability to adjust F₁ and F₂ over the course of a patient's recovery provides the advantageous capability of adjusting the percentage of a patient's weight being borne by the injured foot and ankle in a controlled manner.

FIGS. 8B and 8C show alternative force diagrams for the configuration of the orthopedic bracing system 2 without a PTB 4, as shown in FIG. 8A. In FIGS. 8B and 8C, the downward force from the user's weight is shown as the vector W. The base 66 of AFO 60 supports the force from the user's weight in FIGS. 8B and 8C. The ground reaction force, shown as vector GR, is the upward force on the AFO 60 that occurs when the base of walking boot 36 contacts the ground. The ground reaction force is also distributed over base 66 when the AFO 60 is resting on foot bed 42 of walking boot 36, as shown in FIG. 8B. However, when the AFO 60 is suspended above the foot bed 42, as in FIG. 8C, the ground reaction force is transmitted through the attachment point between the AFO 60 and the walking boot 36.

FIG. 8D shows an alternative attachment mechanism 86 that may be used to connect the AFO 60 to the walking boot 36. One or more tracks 88 may be cut into the upright portion 76 of the AFO 60. The tracks 88 can be configured to attach the back of AFO 60 to the knobs 90 on the interior of upper section 52 in walking boot 36. Other attachment mechanisms 86 are also contemplated. The AFO 60 may be attached to the walking boot 36 by a track, rail, spine, extension, prominence, flush mount bracket, a snap fit connection, a hook and loop connection (e.g.,)VELCRO®), a magnet or a strap. For example, the walking boot 36 may have an attachment mechanism 86 such as a track, rail, spine, extension, prominence or flush mount bracket on an interior surface of the upper section 52 and the AFO 60 may have a channel, slot, groove, recess, depression, receptacle, or flush mount bracket on the exterior surface of the upright portion 76 configured to cooperate with the attachment mechanism 86 on the interior surface of the walking boot 36. The channel, slot, groove, recess, depression, or receptacle on the exterior of the AFO 60 may be configured to capture the track, rail, spine, extension, or prominence of the walking boot 36 to releasably attach the AFO 60 to the walking boot 36. Alternatively, a flush mount bracket, button, knob or lever on the exterior of the AFO 60 may be configured to cooperate with a flush mount bracket or track in the walking boot 36 to detachably connect the AFO 60 to the walking boot 36. Alternatively, the AFO 60 may have a flush mount bracket or track on an exterior surface and the walking boot 36 may have a flush mount bracket, button, knob or lever on its interior surface. The flush mount bracket, button knob or lever on the interior of the walking boot 36 may be configured to cooperate with the flush mount bracket or track on the AFO 60 to detachably connect the AFO 60 to the walking boot 36.

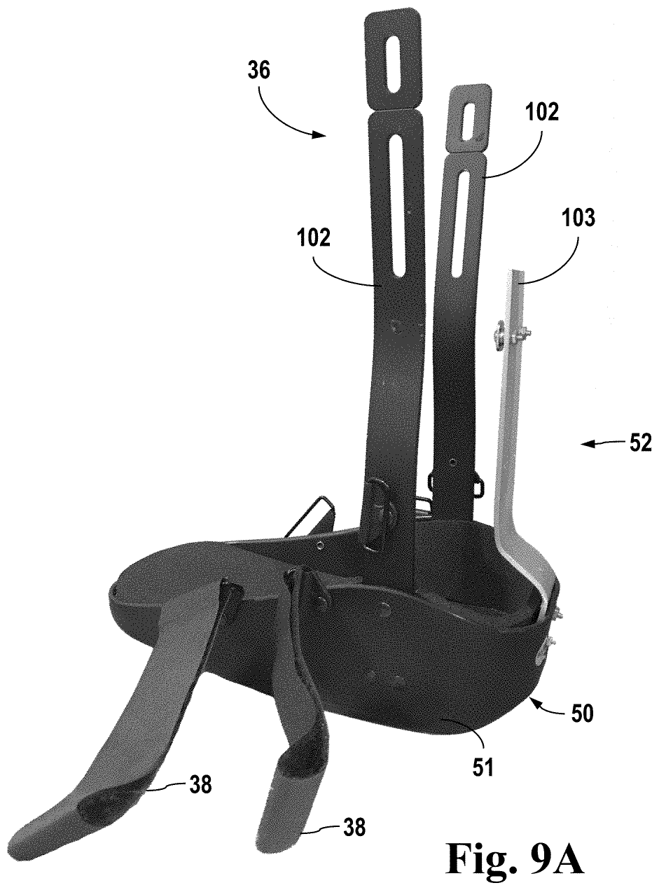

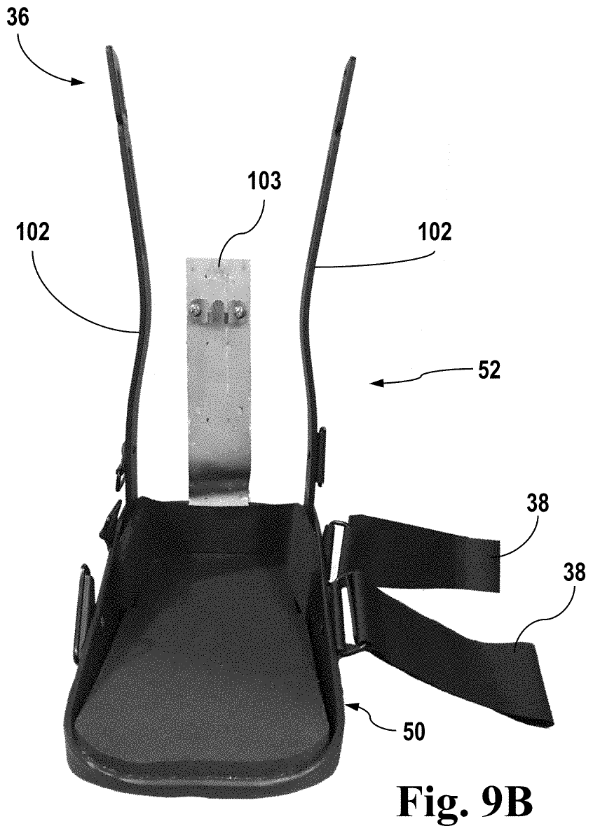

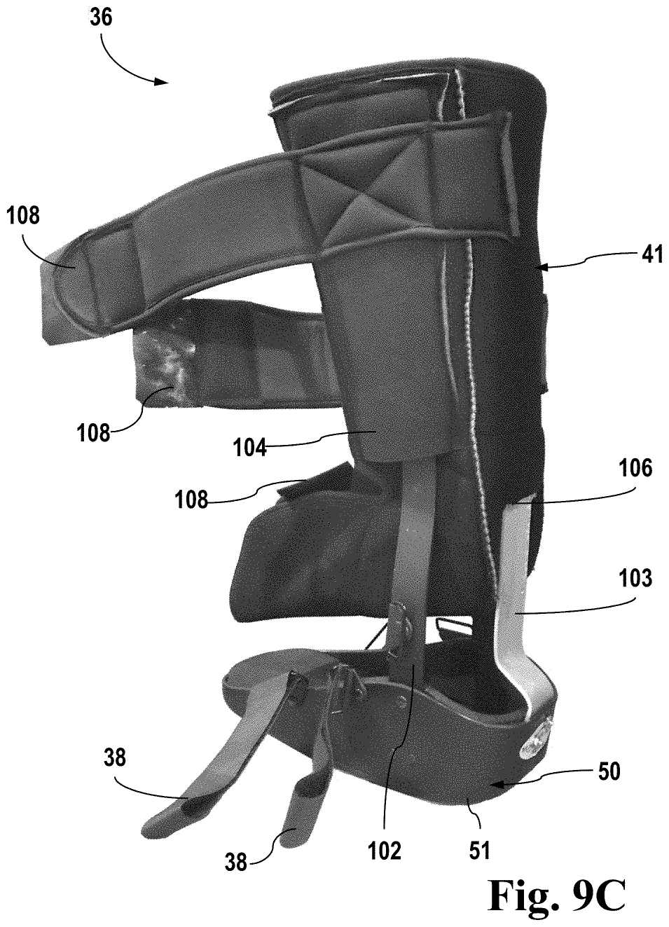

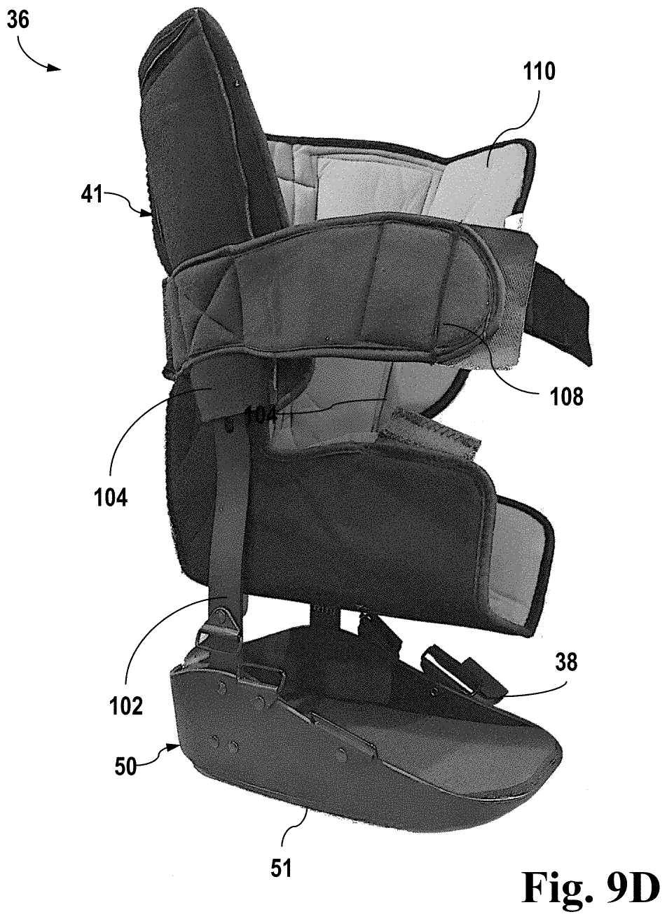

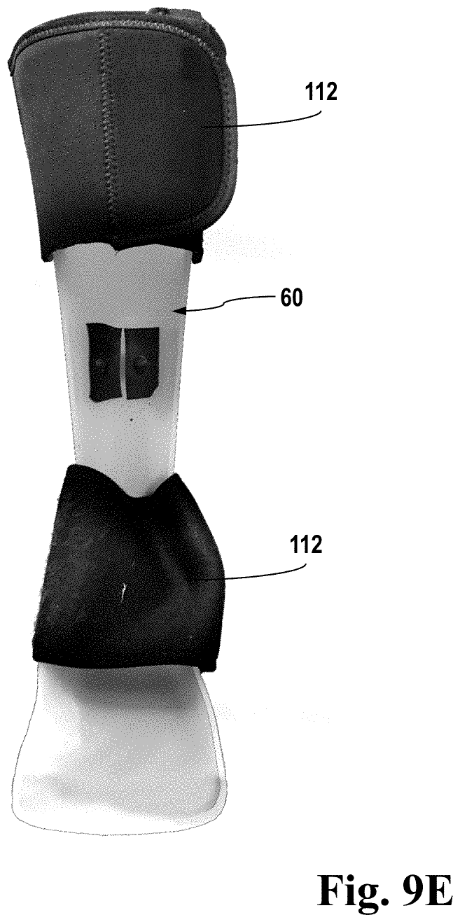

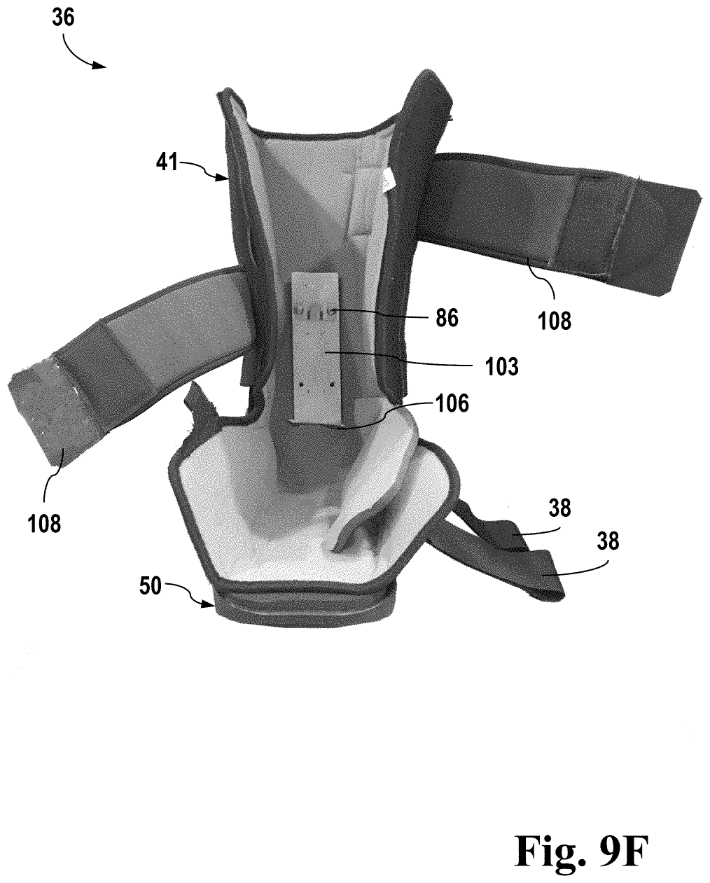

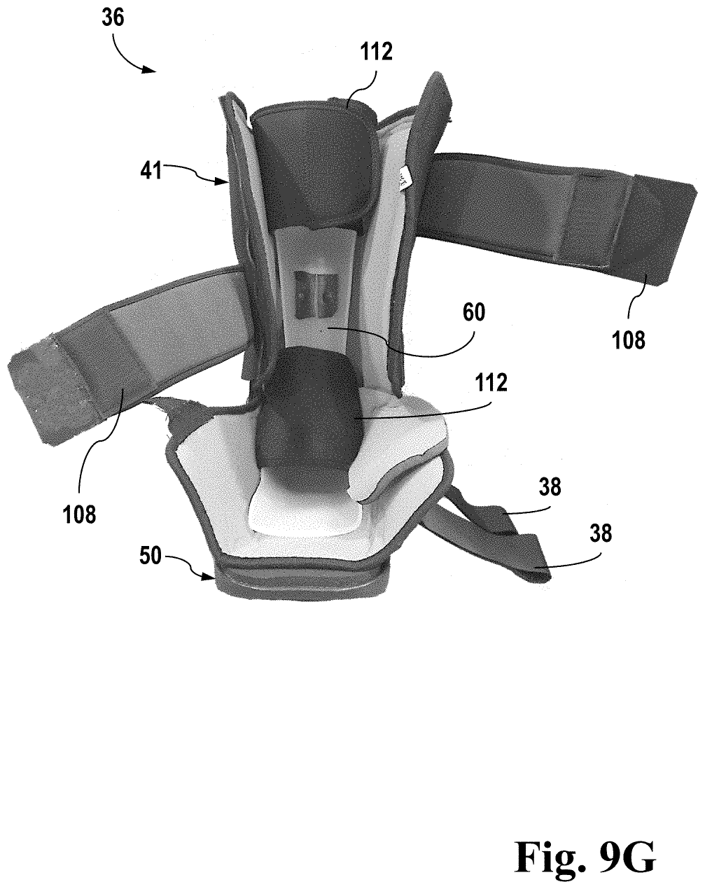

Further details and features of the embodiment earlier described with reference to the walking boot 36 are shown in more detail in FIGS. 9A-9G. As explained above and shown in FIGS. 9A and 9B, the upper section 52 of walking boot 36 may be formed by rigid uprights 102, 103 connected to base 50. The uprights 102, 103 can be spaced apart from each other. For example, there may be one upright 103 that may be positioned near the heel of the user and additional uprights 102 may be located on the lateral sides of the user's leg. The uprights 102, 103 may be formed from any rigid material, e.g., plastic or metal, e.g. steel. For example, the uprights 102, 103 may be formed from any rigid, hard durable material including plastic, metal, steel, aluminum, fiberglass, carbon fiber, composite material or any combination thereof. The uprights 102, 103 may also be formed from, e.g., polypropylene or polyethylene. Other materials are possible, as would be appreciated by those skilled in the art.

The base 50 may be formed from a rigid, durable material such as metal or plastic. The metal or plastic may be covered with padding, e.g., foam padding, fabric or a combination of foam, fabric or other elastomeric material. The sole 51 may be formed from an elastomeric material, such as rubber. Straps 38 can be attached to the base 50 and used to attach the base of the walking boot 36 to the patient.

As shown in FIGS. 9C and 9D, a liner 41 slides over the uprights 102. The liner 41 can have side pockets 104 that open along the bottom edge, allowing the side pocket 104 to slip over an upright 102. The side pockets 104 may be narrower at the bottom to maintain proper liner and foot positioning with respect to the base of the boot. The side pockets 104 may be wider at the top to accommodate larger or smaller legs by adjusting the position of the liner 41 relative to the back of the walking boot 36.

The liner 41 may also have a slit 106 through which the rear upright 102 can slide. When the rear upright 103 passes through the slit 106 leading to the interior of the liner, as shown in FIG. 9F, the attachment mechanism 86 is exposed such that an AFO 60 can be attached to it.

It is also possible to secure the bottom of the liner 41 to the base 50 of the walking boot 36 with, for example, VELCRO®, snaps, hooks or other suitable attachment mechanisms known in the art.

As shown in FIG. 9D, the front flap 110 can be wrapped around the front of the patient's lower leg and the liner 41 can then be secured using wide bands 108. The bands 108 may be made from, e.g., fabric, foam or a combination of materials and attached to the liner 41 at one end to prevent tangling, twisting or loss. The bands 108 can wrap around a portion of the lower leg and liner 41 and the loose ends of the bands 108 may then be secured with, e.g., VELCRO®, snaps, hooks, buttons or another suitable attachment mechanism. Additionally, the bands 108 may be oriented such that they pass around the leg in different directions, i.e., left to right or right to left, to facilitate tightening of the bands 108 and a secure fit.

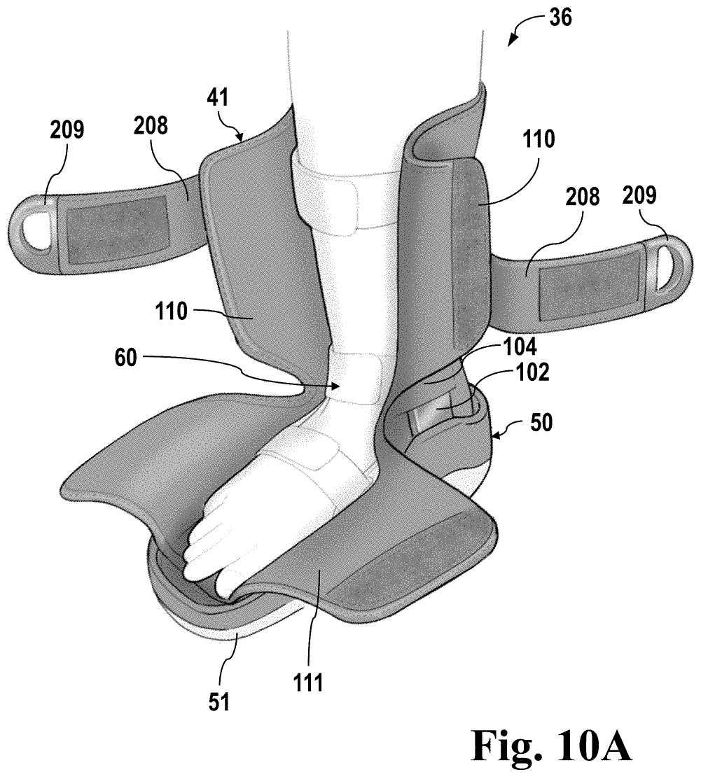

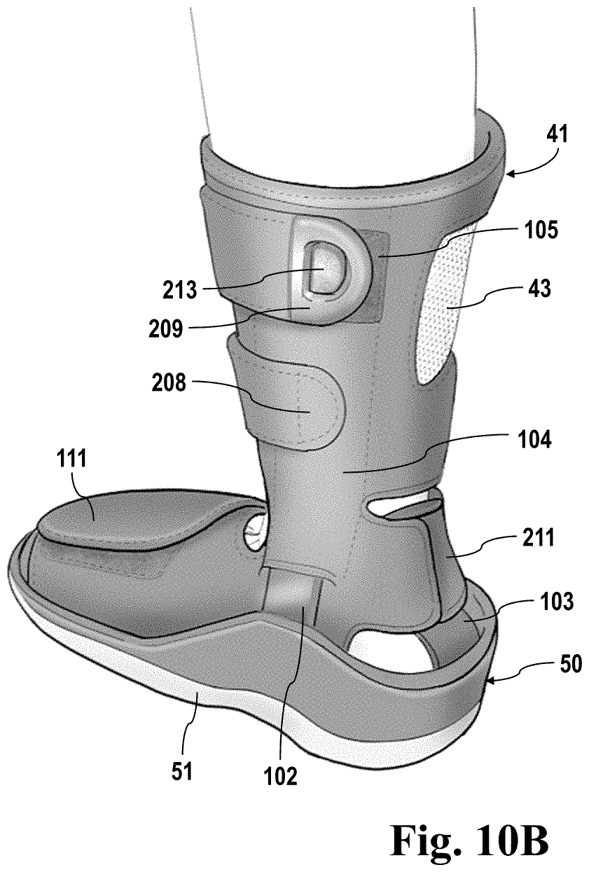

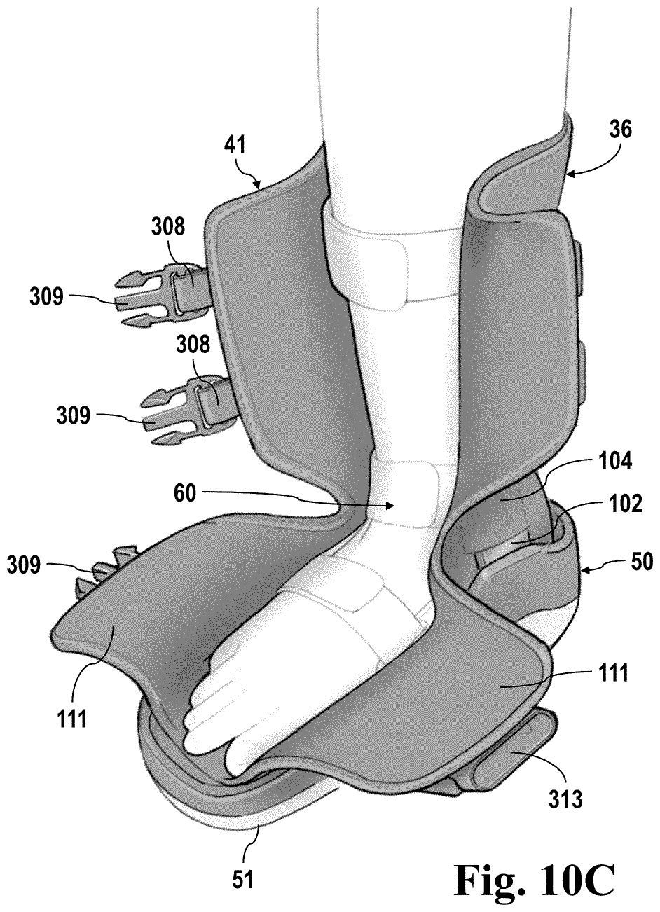

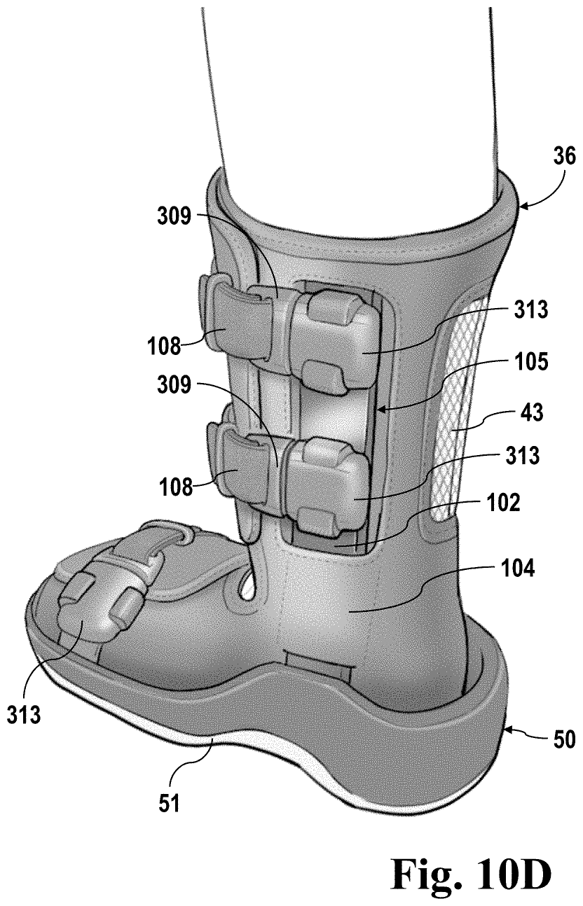

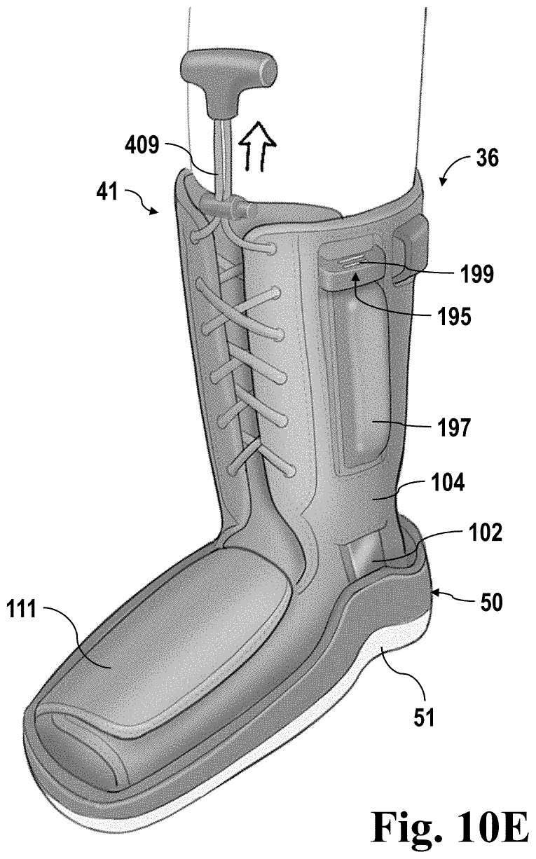

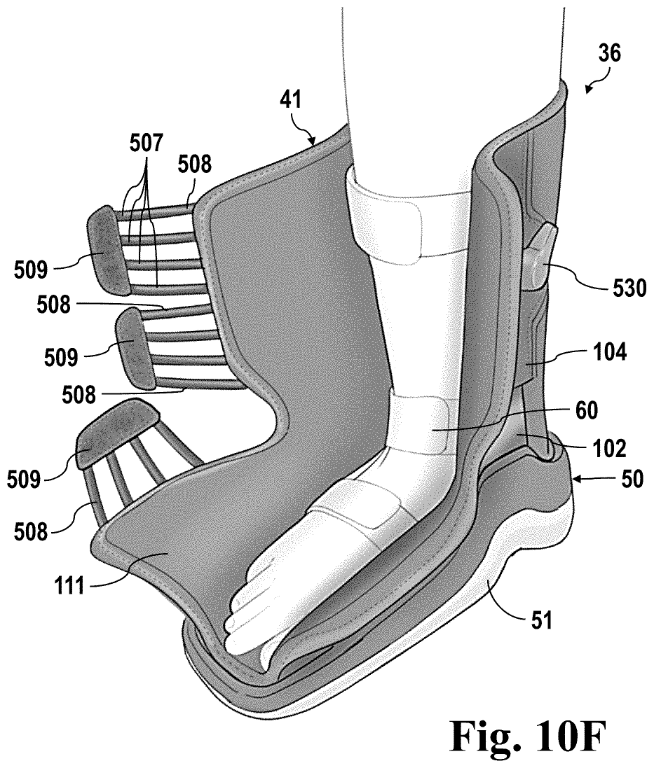

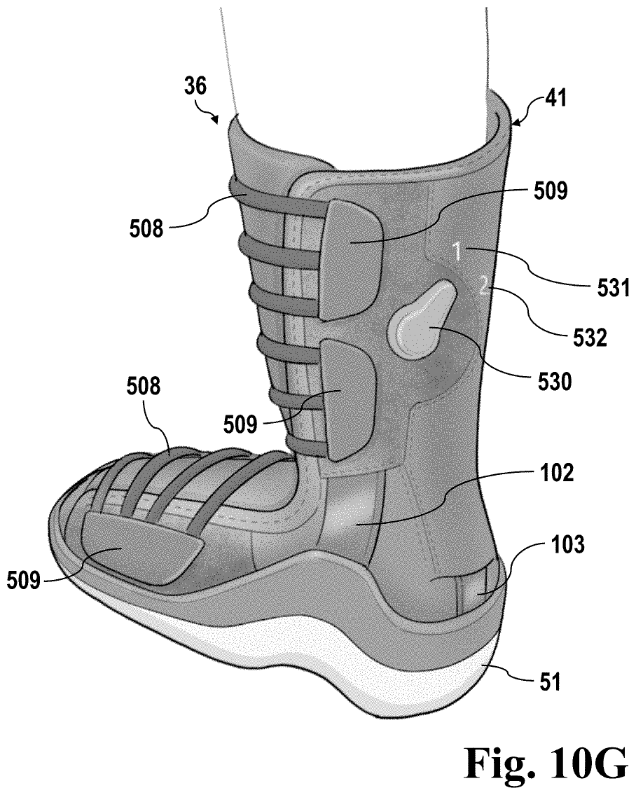

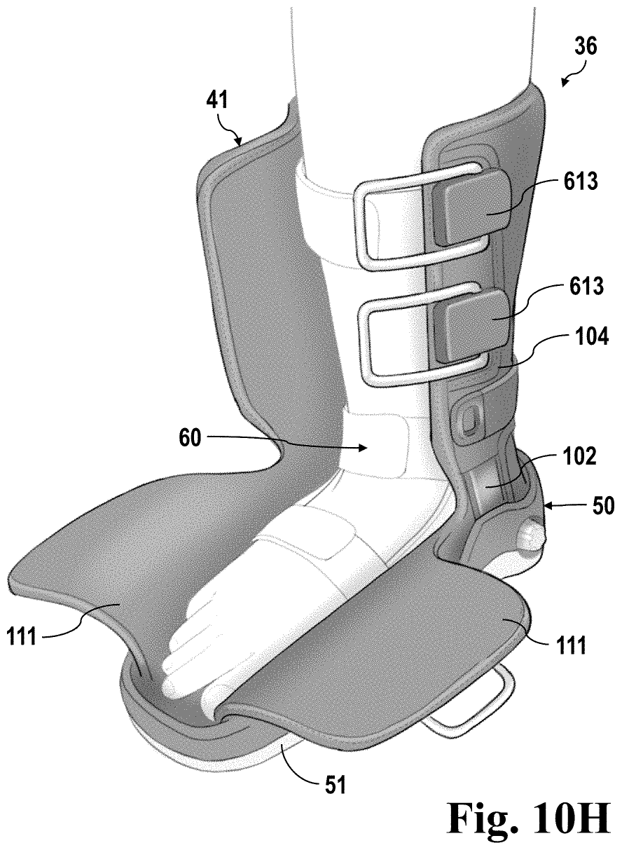

Various methods of securing the liner around the patient's leg are possible. For example, FIG. 10A shows a liner 41 that is secured to walking boot 36 by side pockets 104 that slide over uprights 102, thereby attaching the liner 41 to the boot 36. The liner is then secured to the user's leg using bands 208. As in the example of FIG. 9D, the bands 208 in FIGS. 10A and 10B are attached to the liner at one end. As can be seen in FIG. 10A, the free end of the bands 208 can be a loop 209 that may be made from any suitable material such as, e.g., plastic, fabric, metal, etc. The bands 108 can wrap around a portion of the lower leg and liner 41. The loop 209 may be attached to a knob 213 (see FIG. 10B) that is attached to an upright 102. Side pockets 104 may have an opening 105 such that knob 213 is accessible after liner 41 is secured to walking boot 36. Further, there may be a strap 211 that secures the liner to rear upright 103.

Additional optional features of the liner 41 are also shown in FIGS. 10A and 10B. For example, a toe flap 111 may wrap around the top of the patient's foot. There may also be mesh sections 43 in liner 41 to allow for better air flow and ventilation.

In the example of FIGS. 10C and 10D, liner 41 is secured to walking boot 36 by side pockets 104 that slide over uprights 102 thereby attaching the liner 41 to the boot 36. The liner is then secured to the user's leg using bands 308. The bands 308 may be adjustable. As can be seen in FIG. 10C, the free end of the bands 308 may be connected to a first end of a side release buckle 309. As shown in FIG. 10D, the bands 308 can wrap around a portion of the lower leg and liner 41. The first end of the side release buckle 309 may then be attached to a second end of a side release buckle 313 that is attached to an upright 102. Side pockets 104 may have an opening 105 such that the second end of the side release buckle 313 is accessible after liner 41 is secured to walking boot 36.

In the example of FIG. 10E, liner 41 is secured to walking boot 36 by side pockets 104 that slide over uprights 102 thereby attaching the liner 41 to the boot 36. The liner is then secured to the user's leg using laces 409. The laces 409 may be tied or, as can be seen in FIG. 10E, any known quick lacing system may be used to secure the laces 409.

In the example of FIGS. 1OF and 10G, liner 41 is secured to walking boot 36 by side pockets 104 that slide over uprights 102 thereby attaching the liner 41 to the boot 36. The liner is then secured to the user's leg using bands 508. The bands 508 may be multiple small bands or cords 507. As can be seen in FIG. 10F, one end of multiple small bands or cords 507 may be connected to a first end of a buckle 509 to form a band 508. As shown in FIG. 10G, the bands 508 can wrap around a portion of the lower leg and liner 41. The first end of the buckle 509 may then be attached to a second end of the buckle (not shown) that is attached to an upright 102 or may optionally be attached to the liner 41.

In the example of FIGS. 10H and 10I, liner 41 is secured to walking boot 36 by side pockets 104 that slide over uprights 102 thereby attaching the liner 41 to the boot 36. The liner is then secured to the user's leg using a bail and catch buckle 609, 613. One half of the bail and catch buckle 613 may be connected to an upright 102, as shown in FIG. 10H. The other half of the bail and catch buckle 609 may be connected to liner 41, as shown in FIG. 10I. Liner 41 can wrap around a portion of the lower leg and the bail and catch buckle can be fastened to secure the liner around the user's leg. Side pockets 104 may have an opening 105 such that the second end of the bail and catch buckle 613 is accessible after liner 41 is secured to walking boot 36. Optionally, liner 41 may include a pocket 610 that slides over upright 103 and is secured by band 611.

The AFO 60 shown in FIG. 9E, can be attached to the walking boot 36. The wider straps 112 shown in FIG. 9E secure the AFO 60 to the patient's foot and leg. An attachment mechanism 86 on the back of AFO 60 mates with the attachment mechanism 86 on the walking boot 36, shown in FIG. 9F. The walking boot 36 with the AFO 60 attached is shown in FIG. 9G.

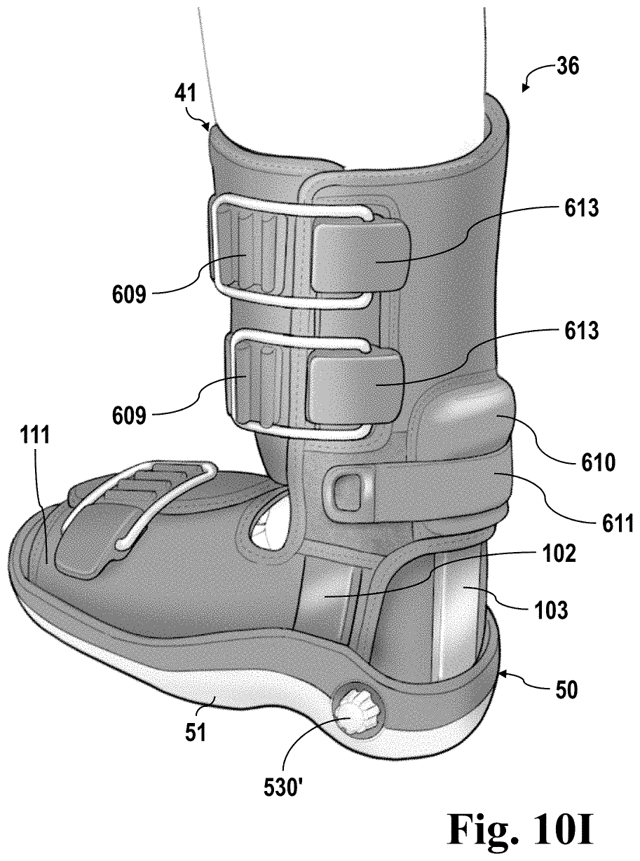

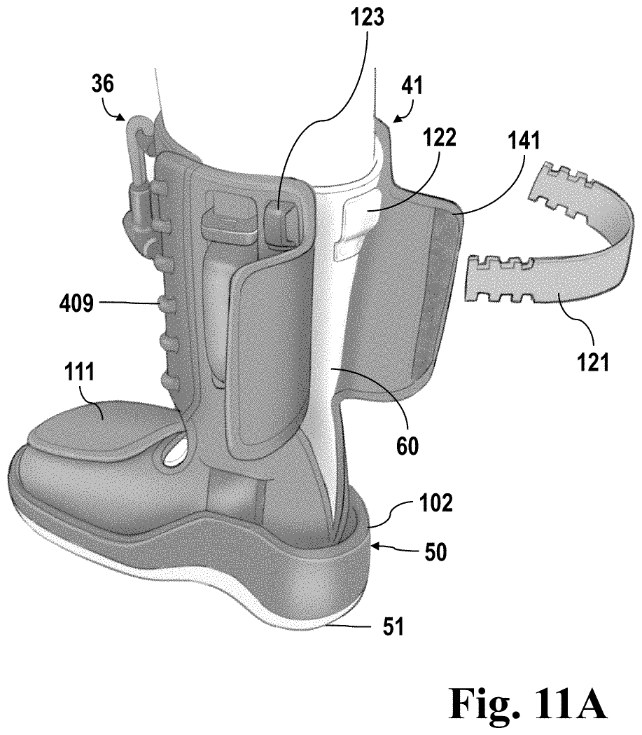

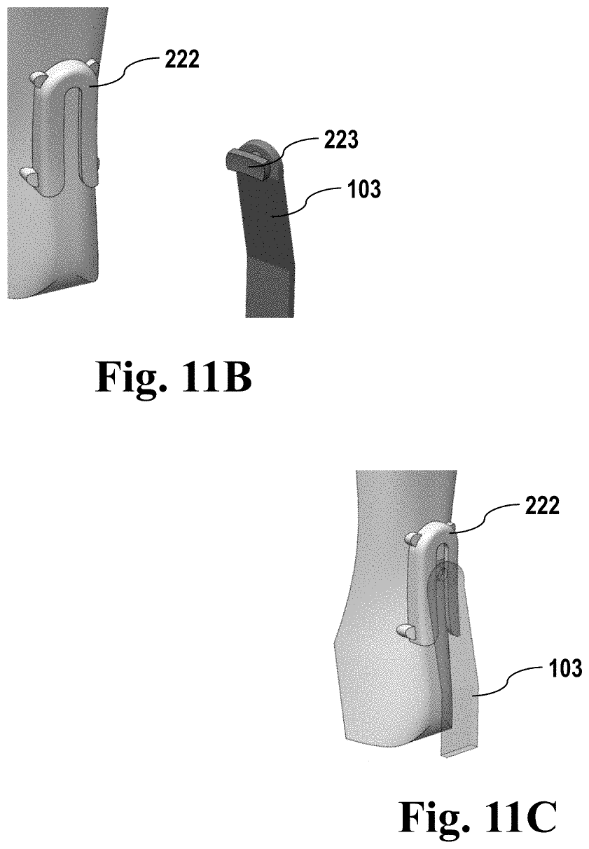

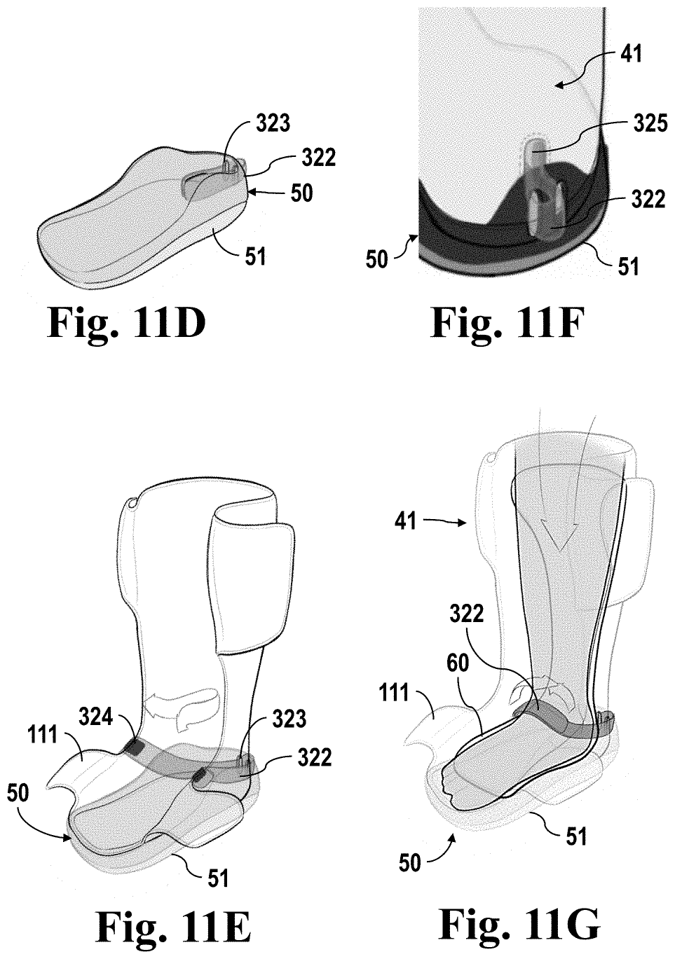

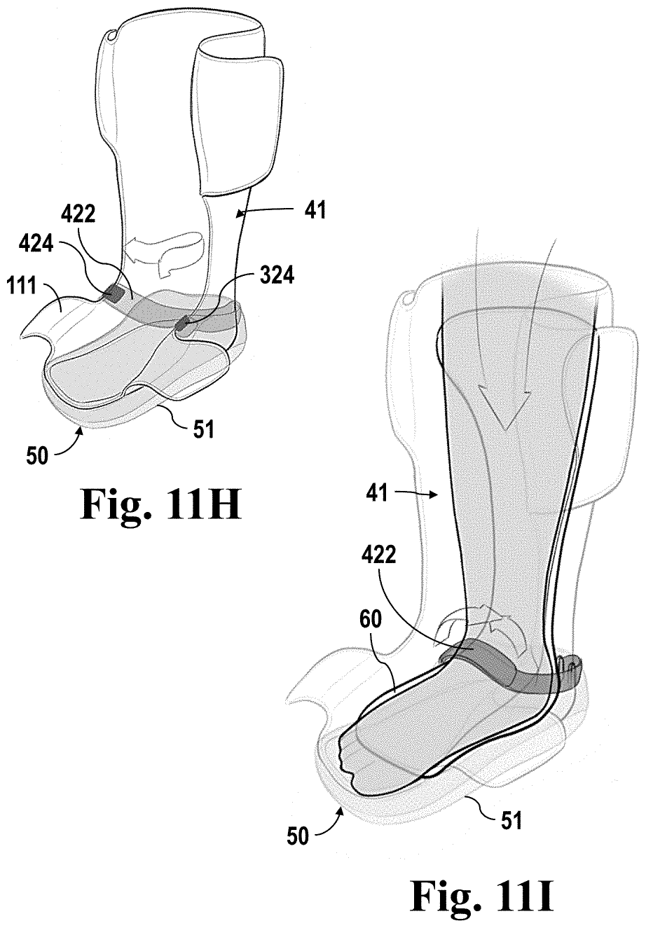

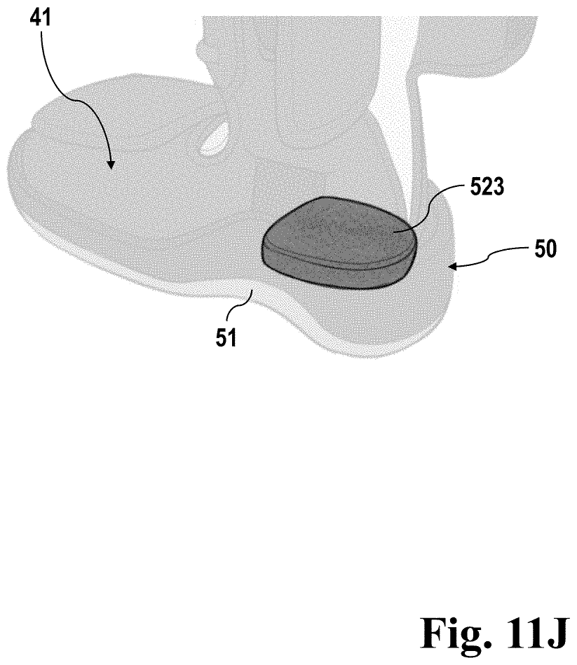

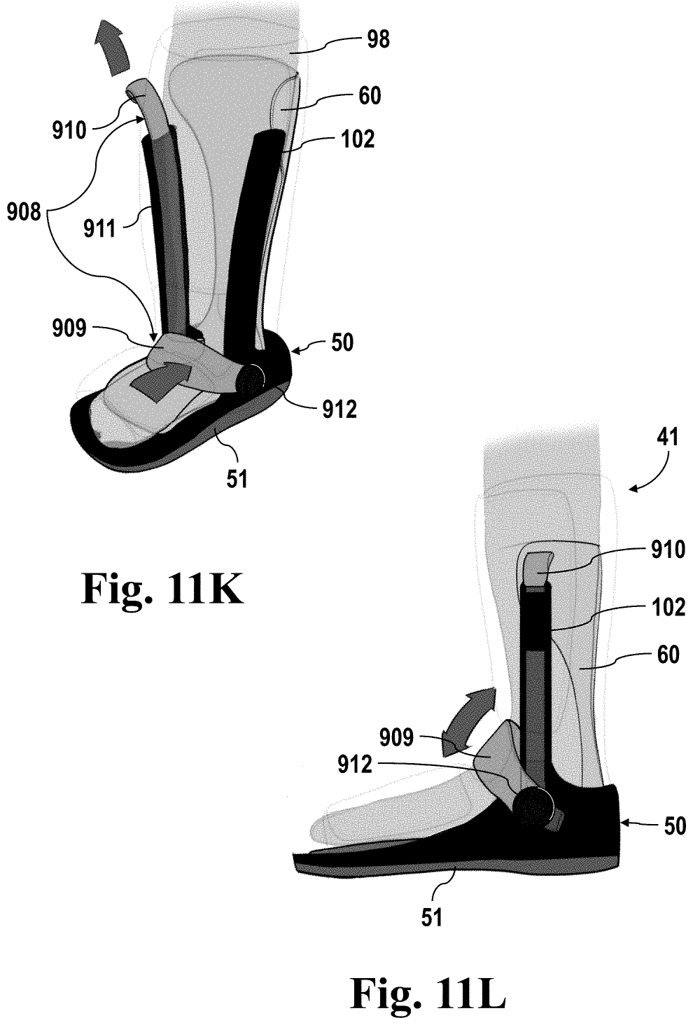

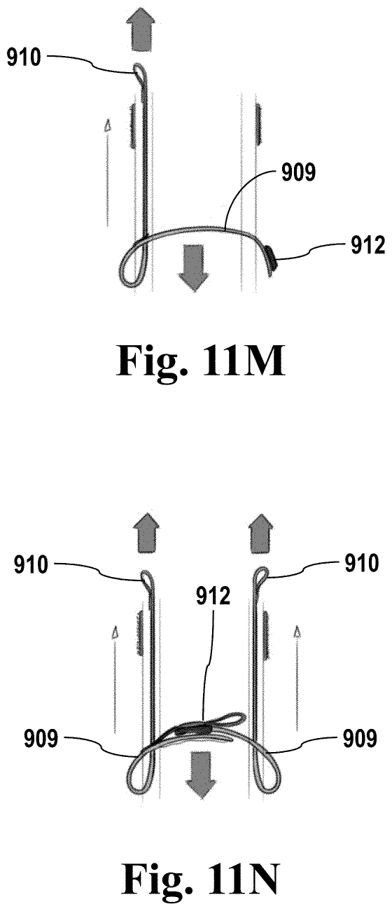

One of skill in the art will appreciate that various ways of attaching the AFO 60 to the walking boot 36 are feasible. For example, as shown in FIG. 11A, there may be an attachment piece 122, such as a hook or a loop, on the back of AFO 60. Liner 41 may have flaps 141 that are configured to open, giving access to the back of AFO 60 and attachment piece 122. There may be a band 121 that passes through attachment piece 122. After passing through attachment piece 122, band 121 may be secured by one or more clasps 123. The clasps 123 may be directly attached to walking boot 36. In the alternative, clasps 123 may be attached to liner 41, which is attached to walking boot 36, thereby indirectly attaching AFO 60 to walking boot 36.

In the example of FIGS. 11B and 11C, the AFO 60 may be attached to walking boot 36 using a knob and track system. As shown in FIG. 11B, a protrusion or knob 223 may be attached to rear upright 103 and a track 222 is attached to the back of AFO 60. AFO 60 may then be attached to walking boot 36 by sliding track 222 over knob 223, as shown in FIG. 11C.

In the example of FIGS. 11D through 11G, the AFO 60 may be attached to walking boot 36 using a strap. Strap 322 may pass through slot 323 in the heel of base 50, as shown in FIG. 11D. Strap 322 may then pass through an opening 325 in liner 41. One of skill in the art will appreciate that strap 322 may be more than a single strap and it may, alternatively, pass through uprights 102 (not shown in FIGS. 11D-11G). The opening 325 may be positioned in the liner to accommodate the placement of the strap 322. The ends of strap 322 may, optionally, also pass through guides (not shown) on the inside of liner 41 to minimize tangling and facilitate placement of the straps relative to AFO 60. The AFO 60 can then be placed into the liner 41, strap 322 can be wrapped around AFO 60 and the user's foot or lower leg and secured using, e.g., a hook and loop closure 324, as shown in FIGS. 11E and 11G. One of skill in the art will appreciate than any suitable closure 324 may be used, including, but not limited to, snaps, buttons, buckles, latches, etc.

Alternatively, as shown in FIGS. 11H and 11I, AFO 60 may be attached to walking boot 36 using straps sown directly into liner 41. Strap 422 may be attached to the liner 41 by any suitable means, such as stitching, bonding, adhesive, hook and loop, etc. The AFO 60 can then be placed into the liner 41, strap 422 can be wrapped around AFO 60 and the user's foot or lower leg and secured using, e.g., a hook and loop closure 324. One of skill in the art will appreciate than any suitable closure 324 may be used, including, but not limited to, snaps, buttons, buckles, latches, etc.

Alternatively, AFO 60 may be attached to walking boot 36 using a magnet 523. One possible placement of magnet 523 in the heel of base 50 is shown in FIG. 11J. However, magnet 523 may be placed anywhere in base 50 or in the uprights 102, 103. Magnet 523 may hold AFO 60 in place while liner 41 is secured around the AFO 60 and the user's foot and leg.

In the alternative shown in FIGS. 11K and 11L, the AFO 60 may be attached to walking boot 36 using a continuous strap 908. Continuous strap 908 may be secured to upright 102 by sleeve 911. Continuous strap 908 passes through sleeve 911, leaving a handle end 910 of continuous strap 908 above sleeve 911 and accessible from outside walking boot 36. The opposite end of strap 908 is the attachment end 909. To attach the AFO 60 to walking boot 36, AFO 60 is attached to the patient's leg and placed inside walking boot 36. Attachment end 909 may then be loosely draped across the patient's ankle and secured to base 50 by, e.g., a buckle, snap, VELCRO®, magnet or any other suitable fastener 912. The continuous strap 908 can then be tightened to secure AFO 60 in place by pulling on handle end 910 (see FIG. 11M).

Other configurations of continuous strap 908 are possible. For example, as shown in FIG. 11N, there may be two continuous straps 908, one attached to each upright 102. The attachment ends 909 of the two continuous straps 908 may be secured to each other by fastener 912. Continuous strap 908 may then be tightened by pulling on either or both of the handle ends 910.

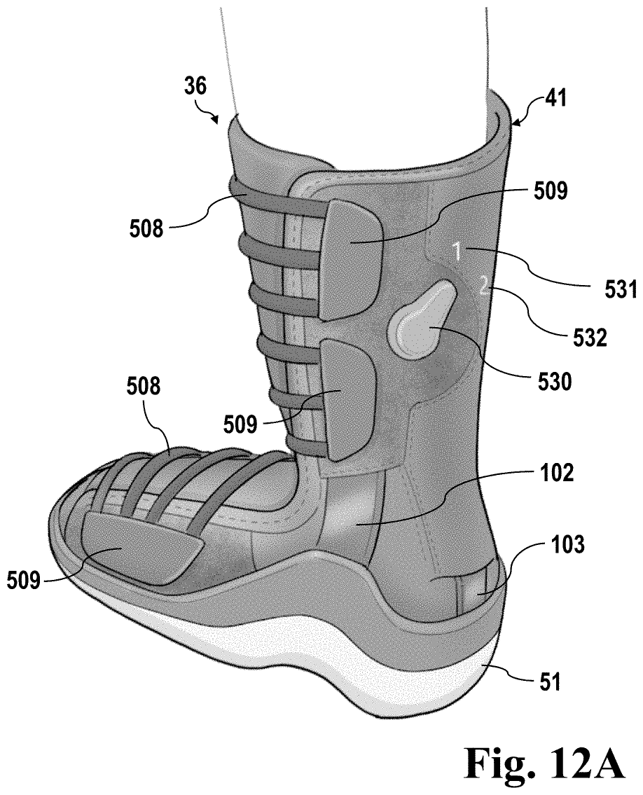

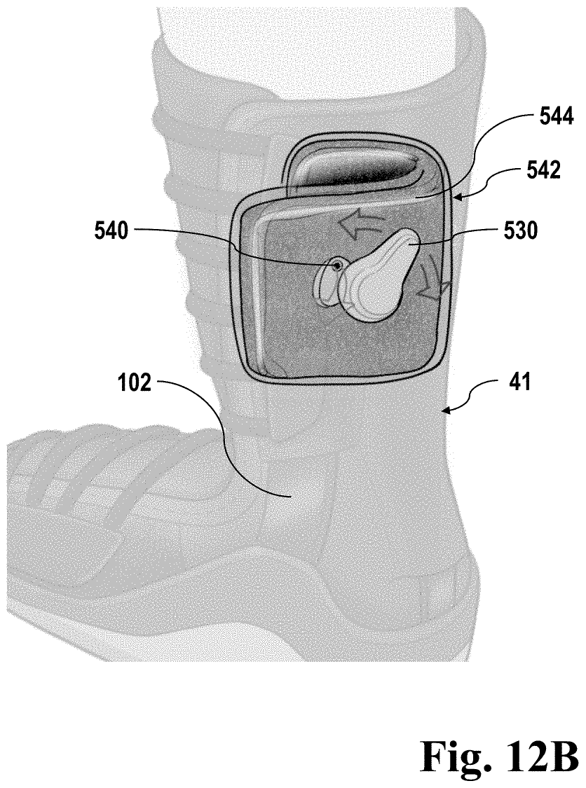

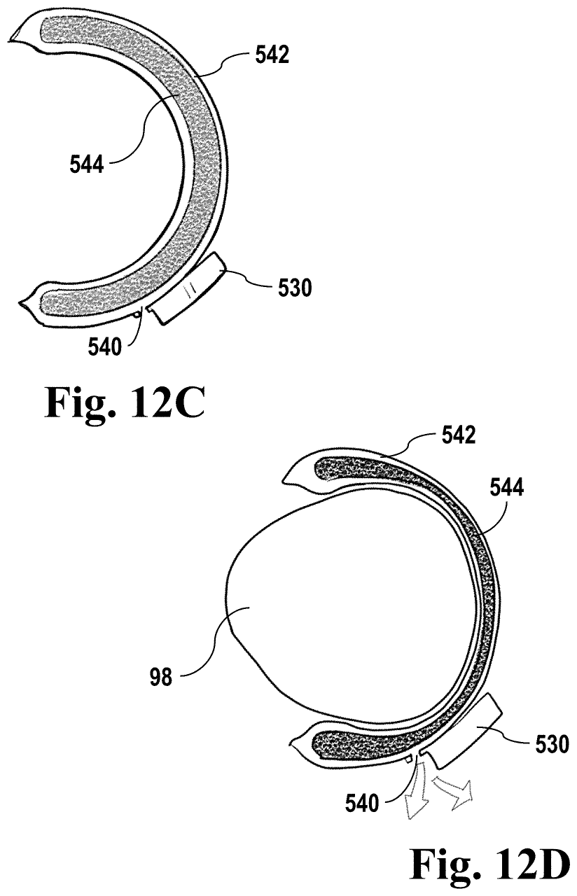

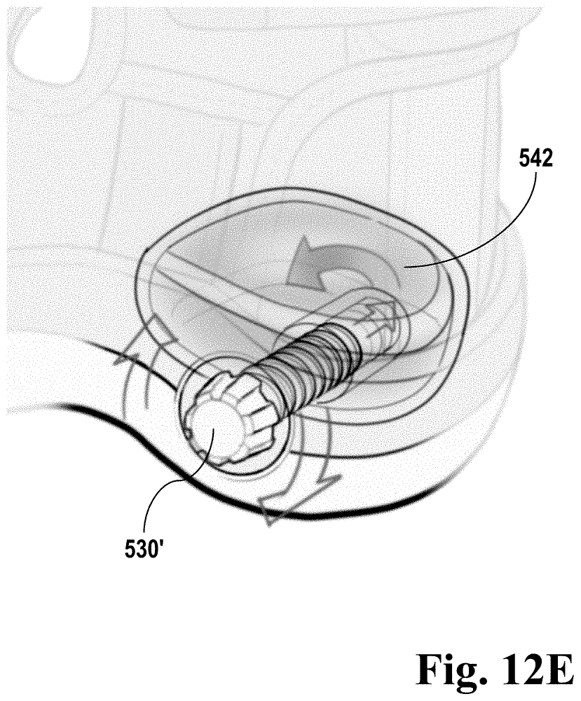

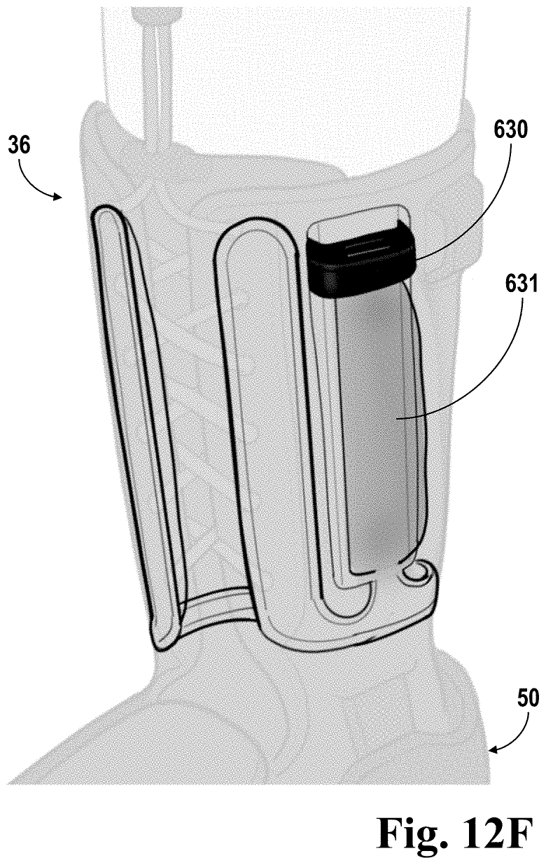

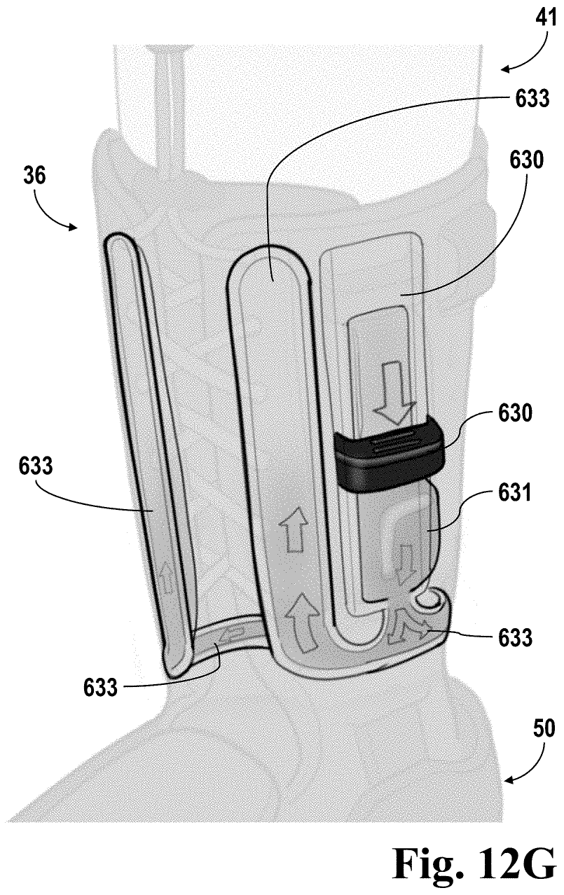

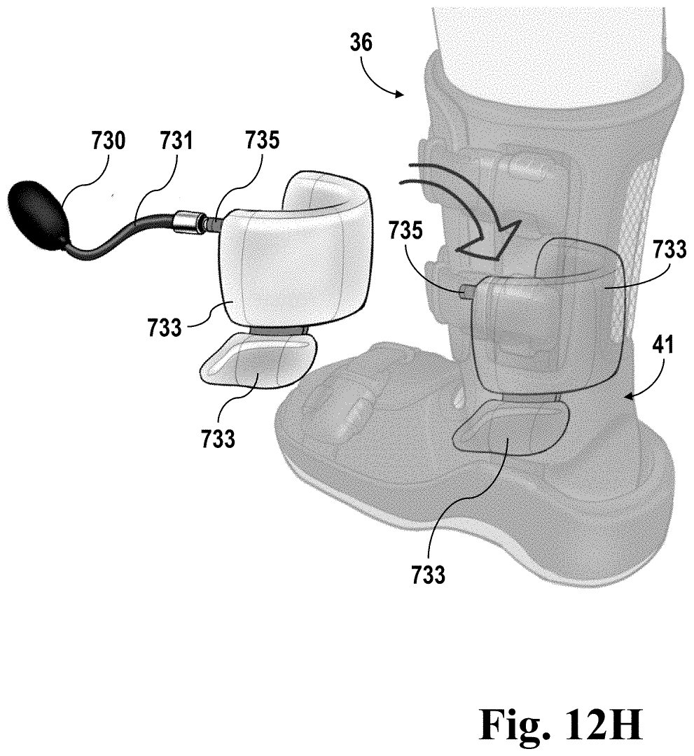

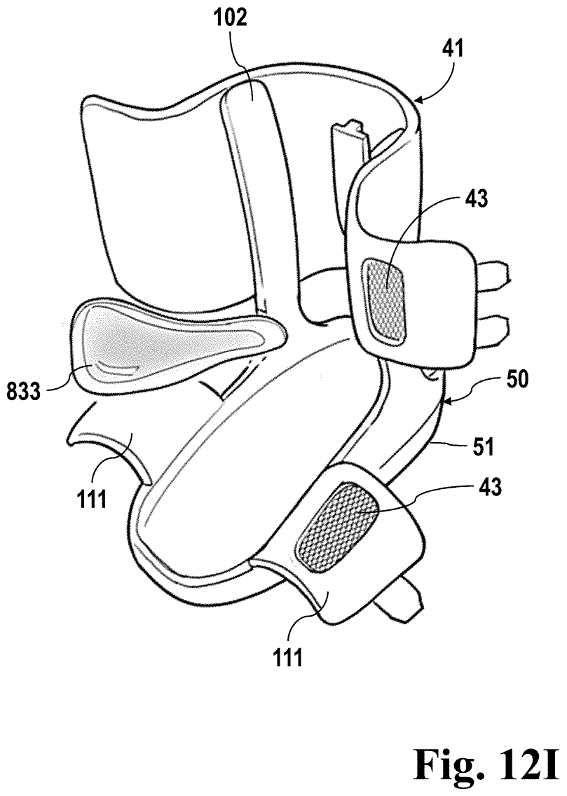

An optional pneumatic device may be added to liner 41 to supply compression as the user's leg is healing. Various configurations of the pneumatic device are possible. For example, a self-inflating bladder, as shown in FIGS. 12A, 12B and 12C, may be used. Liner 41 may have an internal liner bladder 542. Open cell foam 544 may be placed inside liner bladder 542, as shown in FIG. 12B. An air vent 540 may be connected to liner bladder 542. When valve 530 is turned to open position 532, air may flow freely through vent 540 into or out of liner bladder 542. When valve 530 is in closed position 531, no air flows through vent 540 and a relatively constant volume of air is maintained in liner bladder 542, except for the customary degree of leakage in such systems. As shown in FIG. 12C, when liner bladder 542 is empty and valve 530 is turned to the open position 532, air flows through vent 540 into liner bladder 542, inflating open cell foam 544. If valve 530 is left in open position 532 as liner 41 is wrapped around the user's calf 98, as in FIG. 12D, some air will flow out of liner bladder 542 through vent 540. When valve 530 is turned to the closed position 531, the air remaining in bladder 542 and open cell foam 544 will provide compression to the user's calf and ankle.

One of skill in the art will appreciate that different configurations of valve 530 are possible. For example, FIG. 12E shows a screw valve 530′ that may be used to inflate liner bladder 542. Additionally, the screw valve 530′ pushes on the air pocket in bladder 542 and the displacement of air within the bladder 542 creates further compression.

Other configurations of the pneumatic device will work with this disclosure. For example, the pneumatic device may be a pump built into the walking boot 36. A non-limiting example of such a pump is shown in FIGS. 12F and 12G. The pump mechanism shown in FIG. 12F includes a lever 630 and an air intake 631. By moving lever 630 toward base 50, air may be forced from air intake 631 into a liner bladder 633, shown in FIG. 12G.

Another non-limiting example of a pneumatic device that will work with this disclosure is a hand inflation device such as the one shown in FIG. 12H. An inflation bulb 730 may be connected to a channel 731. The channel 731 may be connected to a port 735, which connects to the liner bladder 733. When the inflation bulb 730 is compressed, air flows through channel 731, through port 735 and into the liner bladder 733. Port 735 may be a one-way valve to prevent air from flowing out of the bladder as the compression on inflation bulb 730 is released.

One of skill in the art will appreciate that liner bladders may be configured in various ways, as shown in FIGS. 12A through 12H, depending on the patient's needs for compression. In FIG. 12I, liner bladder 833 is shown in a separate flap. Advantageously, liner bladder 833 may be positioned such that to achieve more direct compression of the ankle. For example, as shown in FIG. 12I, liner bladder 833 may be configured to cover the anterior of the ankle, which is not covered by the AFO 60. Additionally, more than one liner bladder may be used in a liner 41.

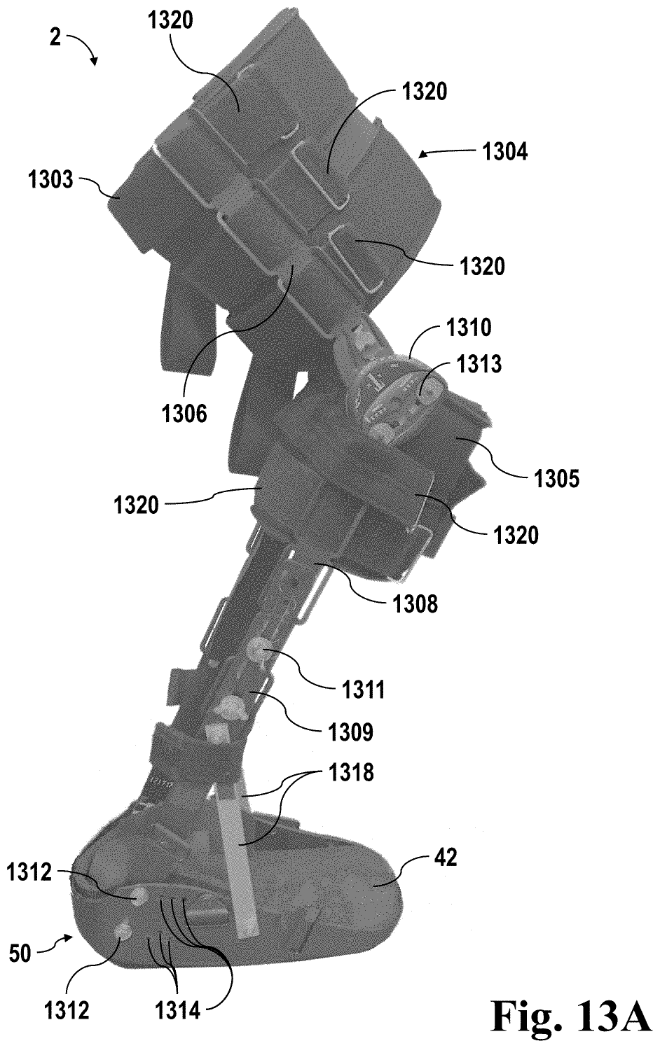

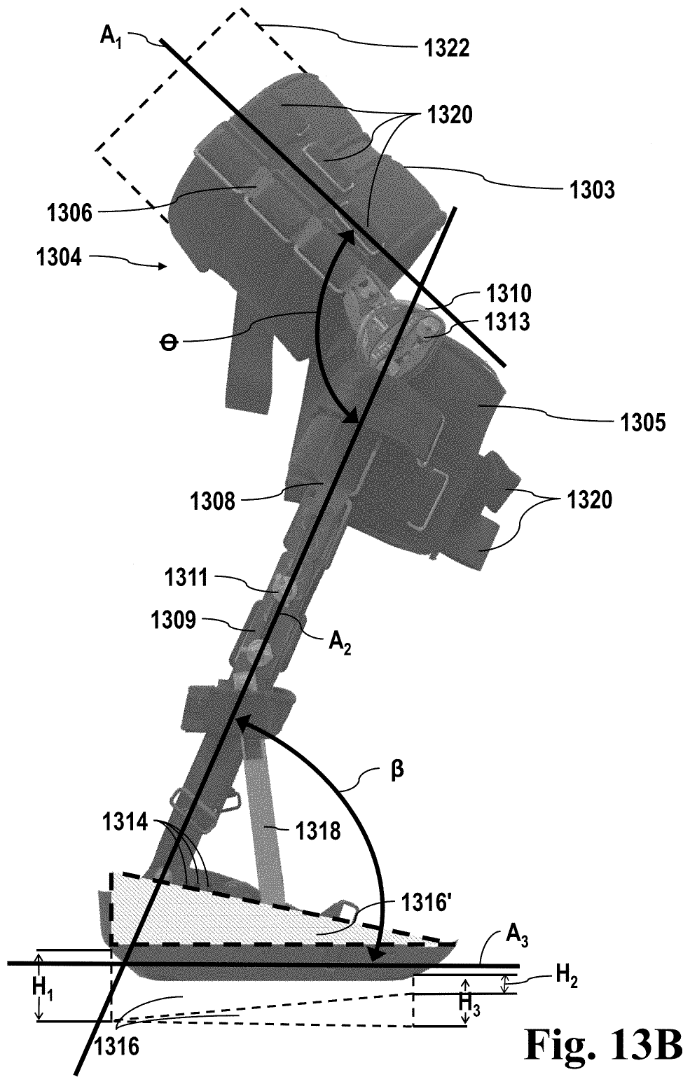

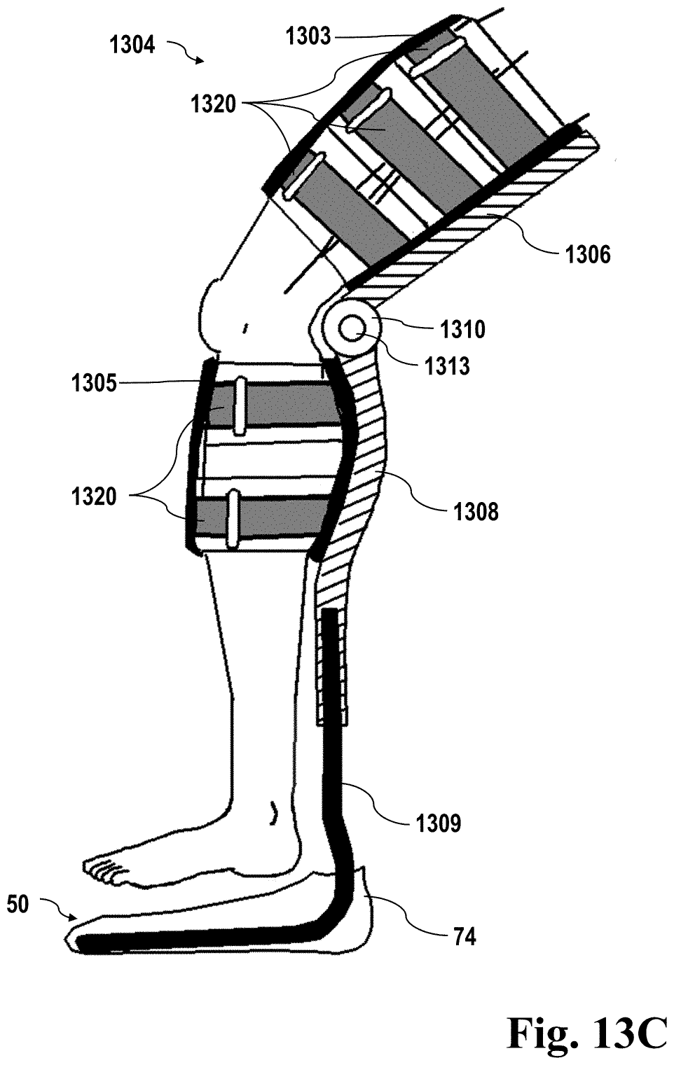

Alternative braces will also work with the previously disclosed embodiments of orthopedic bracing system 2. For example, as shown in FIGS. 13A, 13 B and 13C, the PTB 4 may be replaced with a support 1304. Support 1304 serves the same purpose of reducing or eliminating weight borne by the patient's foot and ankle during recovery and it may be attached to or detached from walking boot 36 during different phases of treatment and recovery. Support 1304 includes a first support portion 1303 surrounding the patient's thigh and a second support portion 1305 surrounding the upper portion of the patient's calf and part of the knee. As shown in FIGS. 13A and 13B, the first support portion 1303 may have rods 1306 that run along the medial and lateral sides of the patient's thigh and straps 1320 that secure the first support portion 1303 to the patient's thigh. The second support portion 1305 may have rods 1308 that run along the medial and lateral sides of the patient's calf and straps 1320 that secure the second support portion 1305 to the patient's calf. Rods 1306 and rods 1308, and thus first support portion 1303 and second support portion 1305, may be pivotably connected by a lockable joint 1310. Further, rods 1308 may be connected to walking boot 36. For example, tracks 1309 may be secured to the base 50 of walking boot 36 by, for example, bolts 1312. Rods 1308 may be inserted into tracks 1309 and secured using fasteners 1311. Additional structural support for tracks 1309 may be provided by, e.g., uprights 1318.

Alternatively, as shown in FIG. 13C, support 1304 includes a first support portion 1303 surrounding the patient's thigh and a second support portion 1305 surrounding the upper portion of the patient's calf and part of the knee. The first support portion 1303 may have a rod 1306 that is positioned along the posterior of the patient's thigh and straps 1320 that secure the first support portion 1303 to the patient's thigh. The second support portion 1305 may have a rod 1308 that is positioned along the posterior of the patient's calf and straps 1320 that secure the second support portion 1305 to the patient's calf. Rod 1306 and rod 1308, and thus first support portion 1303 and second support portion 1305, may be pivotably connected by a lockable joint 1310. Further, rod 1308 may be connected to walking boot 36. For example, track 1309 may extend through an interior portion of base 50 from the area of the toe or, alternatively, the area of the arch and curve to extend upward from the heel 74 of base 50. Rod 1308 may be inserted into track 1309 and secured using fasteners (not shown). Alternatively, rod 1308 and track 1309 may be pivotably connected to facilitate additional adjustments to the positioning of the patient's calf and knee.

Rods 1306 and 1308 may be made from a light-weight material such as carbon fiber, aluminum or hollow tubes made from metal alloys. One of skill in the art will appreciate that alternate materials may be selected for heavier patients.

In such an embodiment, the patient's knee may also be held in a fixed position. For example, as shown in FIGS. 13A and 13B, the joint 1310 may include a lock 1313 such that the rods 1306 and 1308 can be held at a fixed angle θ. Preferably, the patient's knee may be held in a flexed position such that the patient's weight may be borne by a combination of the back of the thigh, the knee and top of the calf.

Support 1304 may be adjusted to redistribute the force from the patient's weight between thigh, knee and foot. For example, in FIG. 13B, axis A₁ runs parallel to rod 1306 and axis A₂ runs parallel to rod 1308. The angle θ is the angle between axis A₁ and axis A₂. The angle θ may be adjusted by unlocking joint 1310, moving the patient's thigh and calf such that the relative positions of rods 1306 and 1308 form the desired angle θ. Joint 1310 can then be locked to maintain the angle θ. The adjustment of angle θ will allow greater or lesser amounts of the patient's weight to be borne by the back of the patient's thigh.

Further, the angle of support 1304 relative to base 50 of walking boot 36 may be adjusted to allow greater or lesser amounts of weight to be borne by the patient's knee. Axis A₃, shown in FIG. 13B, is substantially parallel to base 50 and axis A₂ is substantially parallel to rod 1308. Angle β is the angle between axis A₂ and axis A₃. The adjustment of angle β may be accomplished by moving the bolts 1312 to different attachment points 1314 along the base 50 of the walking boot 36, as shown in FIGS. 13A and 13B. Alternatively, rod 1308 may be pivotably connected to base 50 by a locking joint, not shown.

In most embodiments contemplated herein, an AFO 60 is also detachably connectable to the walking boot 36 (AFO not shown in FIGS. 13A and 13B). Thus, when the angle β is adjusted, the AFO 60 will move relative to the walking boot 36, as discussed above in reference to FIG. 7D. This way, the angle between the patient's foot and calf is preserved when β is adjusted.

Support 1304 may also be adjustable for size. Straps 1320 may be tightened or loosened to accommodate different size thighs. Further, first support portion 1303 of support 1304 may also be adjustable in length to accommodate longer or shorter legs. For example, first support portion 1304 may be extended as shown by dashed line 1322 in FIG. 13B. The same adjustments for size and length can be made to second support portion 1305 to accommodate different size calves.

The position of support 1304 relative to base 50 of walking boot 36 may also be adjusted. In the example shown in FIGS. 13A and 13B, rod 1308 slides into track 1309 and is secured to track 1309 by fasteners 1311. To accommodate longer or shorter legs, rod 1308 may be inserted into track 1309 greater or lesser distances.

Moreover, the sole 51 of walking boot 36 may be adjustable to different heights and angles. For example, wedges 1316 may be releasably attached to sole 51 such that a height of Hi is obtained in the heel of base 50 and, depending on the shape of wedge 1316, a height of H₂ or H₃ is obtained at the toe of base 50. Wedges 1316 may be attached to sole 51 and/or base 50 using plates and screws, adhesives, interlocking tracks, VELCRO®, or any other attachment means known in the art. Alternatively, the height of foot bed 42 may be adjusted by releasably attaching wedges 1316′ to foot bed 42. The density of the material used to make wedges 1316, 1316′ may also be variable, providing adjustable levels of compression as well.

The adjustability of the angles θ and β and the various heights and lengths that may be achieved by adjusting components of support 1304 allows orthopedic bracing system 2 to be fit to any patient such that the patient's hips remain essentially at the same height when walking. In existing non-weight bearing bracing systems, the patient's hip is pushed up when walking, which causes pain in the back and hips.

Support 1304 may replace PTB 4 in the preceding embodiments to reduce or eliminate the amount of weight normally borne on the patient's foot. Support 1304 may also be added to embodiments of this disclosure where PTB 4 is not shown. Just as PTB 4 is removable from orthopedic bracing system 2, support 1304 may be removed when the patient is able to bear his or her full weight on the foot.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A brace assembly, comprising:
a walking boot configured to cover at least a portion of the plantar aspect of a user's foot; and
a support connected to the walking boot, the support having a first support portion configured to be attached to the user's thigh;
wherein the support comprises a lockable joint, wherein when the joint is unlocked, an angular position of the first support portion relative to the walking boot is adjustable, and wherein when the joint is locked, the angular position of the first support portion relative to the walking boot is fixed.

2. The brace assembly of claim 1, wherein the support further comprises a second support portion configured to attach to the user's leg at least partially below the patella.

3. The brace assembly of claim 2, wherein when the joint is unlocked, the position of the second support portion is fixed relative to the walking boot and is adjustable relative to the first support portion.

4. The brace of claim 1, wherein the first support portion has a shape configured to surround the user's thigh.

5. The brace of claim 1, wherein the walking boot has a base axis and a calf axis and an adjustable angle β therebetween, wherein the position of the user's calf relative to the base of the walking boot is adjustable.

6. The brace assembly of any of claim 1, wherein the walking boot has a base whose height is adjustable.

7. The brace assembly of claim 1, further comprising an ankle-foot orthosis (“AFO”) detachably connectable to the walking boot.

8. The brace assembly of claim 7, wherein the AFO is attached to the walking boot by one or more of a rail, a track, a rail and a protrusion, a flush mount bracket, a snap fit connection, a hook and loop connection, a magnet, a strap and a continuous strap.

9. The brace assembly of claim 1, further comprising a pneumatic feature configured to provide compression to the user's foot, ankle and/or lower leg.

10. The brace assembly of claim 9, wherein the pneumatic feature comprises an inflation bulb, a pump and/or a self-inflating bladder.

11. A brace assembly, comprising:
a walking boot configured to cover at least a portion of the plantar aspect of a user's foot;
a support connected to the walking boot, the support having a first support portion configured to attached to the user's thigh; and
an ankle-foot orthosis (“AFO”) detachably connectable to the walking boot.

12. The brace assembly of claim 11, wherein the support comprises a lockable joint, wherein when the joint is unlocked, an angular position of the first support portion relative to the walking boot is adjustable, and wherein when the joint is locked, the angular position of the first support portion relative to the walking boot is fixed.

13. The brace assembly of claim 11, wherein the support further comprises a second support portion configured to attach to the user's leg at least partially below the patella.

14. The brace assembly of claim 13, wherein when the joint is unlocked, the position of the second support portion is fixed relative to the walking boot and is adjustable relative to the first support portion.

15. The brace of any of claims 11, wherein the first support portion has a shape configured to surround the user's thigh.

16. The brace assembly of any of claims 11, wherein the walking boot has a base whose height is adjustable.

17. The brace assembly of claim 11, wherein the AFO is attached to the walking boot by one or more of a rail, a track, a rail and a protrusion, a flush mount bracket, a snap fit connection, a hook and loop connection, a magnet, a strap and a continuous strap.

18. The brace assembly of any of claims 11 further comprising a pneumatic feature configured to provide compression to the user's foot, ankle and/or lower leg.

19. The brace assembly of claim 18, wherein the pneumatic feature comprises an inflation bulb, a pump and/or a self-inflating bladder.

20. The brace assembly of claim 11, wherein the walking boot comprises a foot bed and the distance between the AFO and the foot bed can be adjusted.
